(19) United States
(12) Patent Application Publication
(10) Pub. No.: US 2013/0253179 A1
(43) Pub. Date: Sep. 26, 2013

(54) Functionalization Processes and Reactants Used in Such Processes Using an Isatoic Anhydride or a Derivative Thereof, Biological Molecules Thus Treated and Kits

(71) Applicants: Arnaud Burr, Saint Martin le Vinoux (FR); Ali Laayoun, La Frette (FR); Alain Laurent, Grenoble (FR)

(72) Inventors: Arnaud Burr, Saint Martin le Vinoux (FR); Ali Laayoun, La Frette (FR); Alain Laurent, Grenoble (FR)

(21) Appl. No.: 13/992,049

(22) PCT Filed: Dec. 5, 2011
(86) PCT No.: PCT/FR11/52863

§ 371 Date: Jun. 6, 2013

(30) Foreign Application Priority Data
Dec. 6, 2010 (FR) 1060102

(51) Int. Cl. C07H 21/02

(52) U.S. Cl. CPC C07H 21/02
USPC 536/23.1; 536/25.3; 536/25.32; 536/25.4; 544/94

(57) ABSTRACT

The present invention relates to a process of functionalising at least one ribonucleic acid (RNA) molecule which comprises the following steps:
a) having at least:
   a binding molecule constituted by an isatoic anhydride or a derivative thereof,
   a group of interest, and
   a binding arm linking the binding molecule with the group of interest,
b) reacting the anhydride function of the binding molecule with at least one hydroxyl group in:
   position 2′ of the ribose of one of the RNA nucleotides, and/or
   position(s) 2′ and/or 3′ of the ribose of the nucleotide at the 3′ terminal end of the RNA, and
c) obtaining an anthranilate linking, via the binding arm, the RNA to the group of interest.

The invention also relates to a functionalising reagent able to be used in such processes, a functionalised biological RNA molecule capable of being obtained by these processes and a kit for detecting a target RNA molecule comprising such a reagent.

Said invention finds a preferential application in the field of in vitro diagnosis.

The present invention relates to novel processes particularly of functionalising, labelling, capturing or separating biological molecules, and more precisely natural or synthetic ribonucleic acids (RNA) or synthetic DNA/RNA chimeric nucleic acids. In the remainder of the text, the term “functionalization” or the related terms (“functionalise” for example) will be used, and will equally well be able to mean labelling, capturing, separation or inhibition of biological molecules. They also relate to biological molecules thus treated or labelled, as well as kits which can be used in the field of molecular diagnosis using the detection and analysis of nucleic acids.

The prior art shows that there are many methods of functionalising nucleotides, oligonucleotides or natural or amplified nucleic acids with groups of interest.

A first method consists in fixing the group of interest onto the base, whether this is natural or modified. A second method proposes fixing the group of interest onto the sugar, here too regardless of whether it is natural or modified. The aim of a third method is the fixing of the group of interest onto the phosphate.

The group of interest on the base has been used in particular in the approach of labelling nucleic acids by incorporating directly labelled nucleotides.

Functionalisation on the sugar is generally a lot more neutral than functionalisation performed on the phosphate, which has an impact on the sensitivity or on the base, which affects specificity.

In fact, the person skilled in the art who has to carry out a functionalisation of a nucleotide or of a nucleotide analogue or of a nucleic acid, is inclined to carry out this fixation onto the base or the sugar, which is more convenient and which offers him/her more alternatives. Furthermore, this is what emerges from a study of numerous documents dealing, in particular, with labelling such as, EP-A-0.063.879, EP-A-0.097.373, EP-A-0.302.175, EP-A-0.329.198, EP-A-0.567.841, U.S. Pat. No. 5,449,767, U.S. Pat. No. 5,328,824, WO-A-93/16094 or DE-A-3.910.151 for the base, or EP-B-0.063.879 or EP-B-0.286.898 for the sugar.

More specifically, the functionalisation on the sugar is often used in the case of nucleic probes prepared by chemical synthesis. There is nevertheless a need for groups of interest which are specific to the binding position, and in particular which do not affect the hybridisation properties of the bases which are involved in the formation of the double helix, via hydrogen bonds, and which are also specific to RNA, finally to functionalise equally ribonucleotides, oligoribonucleotides, RNA nucleic acids which are natural or prepared by transcription, the nucleic acids comprising both at least one RNA part and at least one DNA part, by reverse transcription or by enzymatic amplification.

In the case of labelling and in order to make the targets detectable on the DNA chips, it is necessary to fix a label on them beforehand. This is an important step because it alone makes it possible to detect the presence of the nucleic acids and to make a diagnosis. It is therefore important that the labelling technology be extremely reproducible, robust and sensitive. This is directly linked to the quality and effectiveness of the chemical labelling reagent.

Furthermore, in the chemical functionalisation technologies, the group of interest must not affect the hybridisation properties of the nucleic acids.

In the past the Applicant filed a certain number of patent applications on molecules containing a diazo compound:
EP-B-1.383.732 patent filed on 3 May 2002, and granted on 17 Feb. 2010,
EP05/739660.8 application filed on 24 Mar. 2005,
EP08/805,961.3 application filed on 9 Jun. 2008, and
FR08/55190 application filed on 29 Jul. 2008.

By way of background, the diazo-function-based groups of interest are neither chemospecific to RNA nor regiospecific to a particular position. They therefore functionalise DNA and RNA alike. Furthermore, the diazo function is difficult to reconcile with a conjugation with certain cyanines, such as Cy5, for example.

It is important to be able to be more specific in the functionalisation mechanism, for example for the labelling:

A) In the case of DNA chips measuring the levels of mRNA expression, only the RNA must be labelled. In a complex biological sample, DNA and mRNA may be present and therefore labelled concomitantly. By using a non-chemospecific label, this may lead to an increase in the background noise during hybridisation.
B) The label, which is generally used in great excess, must be destroyed or eliminated before the labelled nucleic acids are put into contact with the probes immobilised on the chip. Indeed, if this were not the case, there would then be a risk of labelling the probes, which would lead to a result which would be impossible to interpret. Having a label specific to RNA would therefore make it possible to avoid this problem of labelling the probes due to an excess of non-hydrolysed label.
C) Due to the steric effects, it is less disruptive for the hybridisation between an oligonucleotide probe and a target made up of nucleic acids to label the ends of the target RNA strand (5′ or 3′) rather than inside. The commercial labelling techniques do not permit such labelling regiospecificity. To our knowledge, only the chemical techniques of oxidation with periodate provide this degree of specificity, but they are severely impacted by the complexity of the labelling protocol (more than three to four reagents and two to three purifications before obtaining labelled RNA); see in this regard:
“An oligonucleotide microarray for microRNA expression analysis based on labeling RNA with quantum dot and nanogold probe” by Ru-Qiang Liang Nucleic Acids Research, 2005, Vol. 33, No. 2 e17 in Nucleic Acids Research, 1996, Vol. 24, No. 22 4535-4532, or
“Chemical methods of DNA and RNA fluorescent Labeling” Nucleic Acids Research, 1996, Vol. 24, No. 22 4535-4532. by Dmitri Proudnikov.

In fact, the 3′ ends of RNA can only be regiospecifically functionalised on the basis of extremely specific enzymatic techniques. Such techniques are used, for example, by Affymetrix (Santa Clara, USA) or by Agilent Technologies (Santa Clara, USA) with specific labelling of RNAs at 3′ with the T4 RNA Ligase and a pCp-labeller-type substrate or by other enzymatic techniques described in certain references:
Huang Z. “Selective labeling and detection of specific RNAs in an RNA mixture” Analytical Biochemistry 2003 129-133,
Martin G. “Tailing and 3′-end labeling of RNA with yeast polys (A) polymerase and various nucleotides” RNA 1998 22-230, or
Huang Z. “A simple method for 3′-labeling of RNA” Nucleic Acids Research 1996 4360-4361.

The problem with enzymatic techniques is their sensitivity to the type of substrate (size and sequence of the RNA to be labelled) and of course their cost associated with both the substrate and above all with the enzyme.

Moreover, the recent development of the detection of microRNAs (small RNA oligomers), which may be pathology labels requires integrable, practical and above all regiospecific technologies for labelling RNAs, and particularly for labelling the 3′ end to limit as far as possible the effects of steric hindrance particularly labelled on small-size duplexes. Moreover these small oligomers are labelled very badly by the enzymatic techniques precisely due to their size, as described by F. Natt in the reference: Nucleic Acids Research, 2007, Vol. 35, No. 7 e52.

There is therefore a great benefit in having low-cost chemical functionalisation technologies, which in addition to being specific to RNA would also be specific to the 3′ end, regardless of the size and of the sequence of the substrate to be labelled.

D) Although the diazo labelling technology is an excellent technique, the nature of the synthesis steps which lead to these molecules is not compatible with the chemical nature of certain fluorophores (Cy5 for example, which is unstable in the presence of hydrazine). The diazo technology is therefore preferentially used with biotin as the group of interest. There is a real need to have direct groups of interest bearing a fluorophore, in order to do without supplementary steps, such as the step of detecting biotinylated compounds by a fluorescent streptavidin molecule. It is therefore important to have a versatile technology where the group reacting with the nucleic acids (isatoic anhydride, for example) is chemically compatible with a conjugation with various groups such as Cy3 or Cy5, for example.
E) Finally, the chemoselectivity of RNA vis-à-vis DNA may also have applications in sample preparation, also called “sample prep”, such as the selection of the RNAs without using a DNAase, the selective capture of RNA in a medium containing DNA, the decontamination, the selective inhibition of amplification, etc.

In 1982, Moorman (Moorman JACS 1982 104 6785-6786) published the use of isatoic anhydride for its selective reactivity with the serines of the chymotrypsin, which leads to the inactivation of the protein. This publication is one of the first which demonstrates the reactivity of isatoic anhydride on hydroxyl groups of a biomolecule, in an aqueous medium. However, and curiously, whereas it would be expected to have a preferential reactivity with the amine functions present on the protein, since the latter are more nucleophilic, it has emerged that the alcohol functions react first.

In 1983, Hiratsuka (Hiratsuka BBA 1983 742 496-508) published one of the first articles on the reactivity of isatoic anhydride or methylated isatoic anhydride on free ribonucleosides (5′ triphosphate or 5′-OH). This was in order to synthesise fluorescent substrates of enzymes to study the latter. Indeed isatoic anhydride, once opened, becomes fluorescent. However, to avoid any confusion, it is necessary to differentiate between the intrinsic fluorescence of opened isatoic anhydride, which becomes an anthranilate molecule (Excitation: 335-350 nm and Emission: 427-446 nm), and the fluorescence which is provided by conjugation with a fluorophore. Indeed, there are a multitude of articles citing the labelling of RNA by isatoic anhydride, but using the intrinsic fluorescence of anthranilate to detect it). Their conclusions are as follows:
isatoic anhydride does not react on the exocyclic amines of the bases, despite being generally described as being far more nucleophilic than 2′-OH,
isatoic anhydride is not known to react on 5′-OH,
   Isatoic anhydride reacts preferentially (kinetically) on 2′-OH than on 3′-OH (thermally favoured). However, due to the migration of the acyl groups between positions 2′ and 3′, a mixture of the order of 90% ester at 3′ is obtained.

In 1990, Ovodov (Ovodov, FEBS 1990 270 111-114), through research based on the work of Khorana in 1963 (Stnark, Journal of the American Chemical Society 1963 75 2546 and Knorre Biokhimiya 1965 1218-1224), describes the acylation of messenger RNAs in aqueous medium with acetic anhydride (DMF 5%, 1M sodium acetate, pH 7, 2 to 3 hours at ambient temperature) to protect RNA from the action of RNases. He describes an acylation level of 70-75% which is sufficient to inhibit the action of the RNases.

In 1993, Servillo (Servillo Eur. J. Biochem. 1993 583-589) published an article demonstrating the “labelling” specific at 3′, of transfer RNA, after incubation with isatoic anhydride (Molecular Probes, Eugene, USA) in aqueous medium and at a pH of 8.8 for 3 hours and at ambient temperature. It demonstrates, using various techniques, an absolutely regiospecific functionalisation at 3′. Through total hydrolysis with the RNase A and the RNase T2, it shows the presence of a single fluorescent nucleoside. With the inhibition of phosphodiesterase, it demonstrates a total labelling in position 3′, corresponding to a regiospecific labelling, not to mention labelling at 5′.

In 2000, there appeared the first article in an ongoing series by K. M. Weeks on selective acylation of the hydroxyls at 2′ of RNA. This series of articles considers the results from Servillo in terms of the regiospecificity of the acylation, since Weeks this time describes a selective acylation of position 2′OH of RNA. It covers the literature on the SHAPE (Selective 2′-Hydroxyl Acylation and Primer Extension) technique and in particular includes the articles:
K. A. Wilkinson, S. M. Vasa, K. E. Deigan, S. A. Mortimer, M. C. Giddings and K. M. Weeks, Influence of nucleotide identity on ribose 2′-hydroxyl reactivity in RNA. RNA 15, 1314-1321 (2009).
K. E. Deigan, T. W. Li, D. H. Mathews and K. M. Weeks, Accurate SHAPE-directed RNA structure determination. Proc. Natl. Acad. Sci. USA 106, 97-102 (2009).
S. A. Mortimer and K. M. Weeks, Time-resolved RNA SHAPE chemistry. J. Am. Chem. Soc. 130, 16178-16180 (2008).
C. M. Gherghe, S. A. Mortimer, J. M. Krahn, N. L. Thompson and K. M. Weeks, Slow conformational dynamics at C2′-endo nucleotides in RNA. J. Am. Chem. Soc. 130, 8884-8885 (2008).
S. A. Mortimer and K. M. Weeks, A fast acting reagent for accurate analysis of RNA secondary and tertiary structure by SHAPE chemistry. J. Am. Chem. Soc. 129, 4144-4145 (2007).
K. A. Wilkinson, E. J. Merino and K. M. Weeks, Selective 2′-hydroxyl acylation analyzed by primer extension (SHAPE): quantitative RNA structure analysis at single nucleotide resolution. Nature Protocols 1, 1610-1616 (2006).
K. A. Wilkinson, E. J. Merino and K. M. Weeks, RNA SHAPE chemistry reveals non-hierarchical interactions dominate equilibrium structural transitions in tRNA^{Asp} transcripts. J. Am. Chem. Soc. 127, 4659-4667 (2005).
E. J. Merino, K. A. Wilkinson, J. L. Coughlan and K. M. Weeks, RNA structure analysis at single nucleotide resolution by Selective 2′-Hydroxyl Acylation and Primer Extension (SHAPE). J. Am. Chem. Soc. 127, 4223-4231 (2005).
S. I. Chamberlin and K. M. Weeks, Mapping local nucleotide flexibility by selective acylation of 2′-amine substituted RNA. J. Am. Chem. Soc. 122, 216-224 (2000).

The author uses derivatives of isatoic anhydride (N-methylated isatoic anhydride, isatoic anhydride, N-methylated nitro isatoic anhydride, nitro isatoic anhydride made to react on transfer RNA or short model oligoribonucleotides (aqueous medium, pH 8, ambient temperature or 37° C., for several hours). Only the least restricted instances of 2′-OH, i.e. least engaged in secondary or tertiary structures, are acylated, because they are more accessible and less close to a phosphate diester backbone. They therefore become more reactive. This partially acylated RNA is then subjected to an in vitro transcription which generate DNA fragments of more or less the same length, the elongation terminating whenever a voluminous 2′-O-anthranilate, i.e. an opened isatoic anhydride molecule which is fixed onto the 2′-OH of the sugar, is encountered. After sequencing or analysis of these fragments by capillary electrophoresis, it is possible to establish a map of the tertiary structure of the messenger RNAs thus submitted to this technique. This is the principle of the SHAPE technique (Selective 2′-Hydroxyl Acylation analyzed by Primer Extension).

In 2008 an article by Li, “Aptamers that recognize drug-resistant HIV-1 reverse transcriptase” Na Li, Nucleic Acids Research, 2008, Vol. 36, No. 21 6739-6751, cites the works by Weeks to specifically internally label the 2′-OHs which would be the most accessible, and thus describe a structural map of the RNA in question.

In 2007, Thomas Cech (Cech T. R. RNA 2007 536-548) also uses this technique to study the structure of crystallised RNAs.

The prior art also includes patents.

The U.S. Pat. No. 7,244,568 relates to more or less partial selective acylation of the 2′-OHs of RNA with hydrophobic groups (butyryl or pentanoyl from the corresponding anhydrides). The RNA thus becomes sufficiently hydrophobic to be selectively extracted by an organic solvent, or be selectively immobilised (for example on a reverse phase, silica phase, a membrane, etc.). It also describes a solid phase activated by an acid chloride or an anhydride which makes it possible to selectively immobilise the RNA molecules relative to the DNA molecules. This phase may be performed by immobilised isatoic anhydride or BCPB (Benzyl chloride immobilised on polystyrene). Finally, it also describes a technique for assaying RNAs adsorbed on a solid phase: the 2′-OHs of the immobilised RNAs are reacted with isatoic anhydride, and the generated intrinsic fluorescence makes it possible to assay immobilised RNA.

More advanced studies demonstrate that in the conditions advocated by this patent, DNA is functionalised in the same way because the anhydrides which it uses are not chemospecific and attack the exocyclic amines of the bases just as much as the hydroxyl groups of the sugar.

The patent EP-B-1.196.631, proposes acylating agents compatible with a regiospecific acylation of the RNA in aqueous medium. These acylating agents do not have to provide an excessively large steric obstruction in order to retain the hybridisation properties of the thus modified RNA. It is principally acylating agents enabling the introduction of an acetyl or formyl group which are used. The thus modified RNA then partially acts as a matrix for a polymerisation reaction; it may also act as a probe in a Northern blot reaction. The idea is to retain the hybridisation properties (the modified RNA remains the substrate of an elongation reaction), whilst destroying the secondary structures through the presence of the group at 2′ and rendering the thus modified RNA resistant to nucleases. Moreover example 22 indicates that the fluorescent labelling of RNA is envisaged, but only by adding methylated isatoic anhydride, meaning that it is an intrinsic fluorescence.

The patent EP-B-1.196.63i proposes a polynucleotide comprising mRNA, rRNA or viral RNA, more than 25% of the riboses of which are modified covalently at the 2′-OH positions. Moreover it relates to a method of producing double-stranded oligo- and polynucleotides from a starting nucleic acid strand, based on multiple mononucleotides (UTP, dTTP and/or dUTP, ATP and/or dATP, GTP and/or dGTP, and CTP and/or dCTP), in the presence of polymerase and possibly primers allowing the formation of a nucleic acid strand complementary to the starting nucleic acid.

The patent application WO-A-2004/013155 describes the chemical modification of RNA in a mixture of the type faeces, blood, etc., in order to differentiate DNA from RNA. Acetic anhydride is the reagent capable of performing this differentiation. The ester function is then hydrolysed to regenerate the biologically active RNA. To do so, this application proposes to promote the use of organic bases which are “just barely” aggressive on RNA (lysine, diamines etc.), associated with a deprotection protocol.

From these documents overall, it should be noted that only the intrinsic fluorescence of isatoic anhydride once opened is used. Moreover the use of this molecule and of its derivatives for other applications is not described as proposed by the present invention.

As far as the fluorescence of isatoic anhydride is concerned, there is little advantage in only having the fluorescence of isatoic anhydride to be able to detect the RNAs. Thus, this fluorescence is weak compared to other fluorescent compounds and not necessarily compatible with the wavelength of the lasers currently used in detection machines. Moreover, with the intrinsic fluorescence of opened isatoic anhydride, the applications are limited to monoplex detection and not multiplex detection as is more and more the case in molecular biology. For example, the labelling for applications in the field of DNA chips is not possible with these patents because the user will not be able to differentiate a detection of a first type of nucleic acids from that of a second type of nucleic acids, both labelled with isatoic anhydride.

In the prior art, there are overall four different uses of isatoic anhydride. The first is to exploit the intrinsic fluorescence properties of anthranilic acid esters to study the mechanism of certain enzymes. The second is the use of the acylating properties of isatoic anhydride to regioselectively inhibit the polymerisation of the nucleic acids, therefore involving only the introduction of a voluminous substituent at 2′ under the mildest possible conditions and in a more or less controlled manner.

Thirdly, the prior art uses isatoic anhydride to prevent their degradation during an extraction step.

The fourth and final use is to selectively extract the acylated RNA from the unacylated DNA. There is therefore an idea of reversibility of the ester function, so as to regenerate a free 2′OH function and therefore a functional RNA.

The idea of conjugating the isatoic anhydride molecule to the biotin or to a Cy3 for applications in the labelling of mRNA for hybridisation on at least two different spots or on a DNA chip is therefore not obvious, because the conjugation with a group of interest may entail a modification of the acylating properties and even of the stability of the isatoic anhydride, which are properties that are difficult to predict. In particular, the conjugation of isatoic anhydride to the group of interest must occur via atoms and bonds which may cause a very substantial destabilisation of the structure, a very great deal of difficulty in the synthesis, poor solubility in water, a loss of the physicochemical properties of the group of interest through attenuation of the fluorescence, a very poor reactivity to RNA, a very great deal of instability of the duplexes formed between the labelled RNA and the DNA (capture probes), in particular by excessive steric obstruction or due to the fact that the functionalised RNA/DNA hybrids are no longer polymerase substrates.

The inventors have furthermore understood the importance of the functionalisation of RNAs by the isatoic anhydride compounds in order to allow them to be captured by recognition molecules, which may or may not be borne by a solid support.

The present invention proposes using isatoic anhydride, not for its intrinsic fluorescence but for its ability to fix specifically onto RNA and to bind with a group of interest, such as defined below.

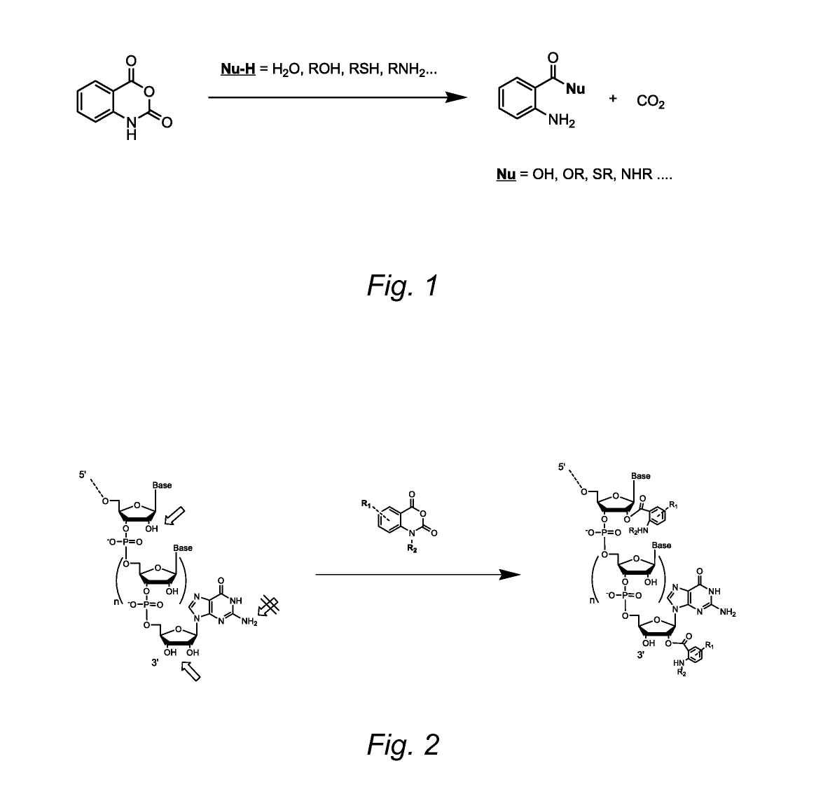

The present invention therefore consists in using an isatoic anhydride molecule, which has been known for a long time (JOC 1959 Staiger 24 1214) for its ability to react with the nucleophiles (primary or secondary amines, thiols and alcohols) to give, after the opening of the cycle, and release of CO₂, derivatives of anthranilic acid (see FIG. 1). The attack of nucleophiles on isatoic anhydride has been widely studied because it allows the direct formation of intermediates which are crucial to the synthesis of several heterocycles of therapeutic interest. Indeed, the derivatives of anthranilic acid may subsequently be rearranged to give rise to a multitude of heterocycles of pharmaceutical interest. Combined with a reactivity vis-à-vis electrophiles (substitution on the benzene ring), isatoic anhydrides thus give access to a very large number of analogues and derivatives used in a great variety of fields of application: agrochemistry, pharmaceutical industry, chemicals and cosmetics industry. Its quite particular nature makes it a relatively stable molecule, which may be sold in dry storage and at ambient temperature, which only reacts in the presence of nucleophiles, preferentially with heat (60-80° C.) and most often in the presence of a base.

These properties, as well as the prior art on the reactivity of isatoic anhydride vis-à-vis alcohols (see above), have led the inventors to think of using this molecule to label and detect RNAs, particularly via a DNA chip. Thus there are few methods which make it possible to functionalise or selectively label RNA, entirely irrespective of the size and the sequence of the substrate. It turns out that the major difference between DNA and RNA is due to the presence of a hydroxyl group in position 2′ (see FIG. 2). This 2′-hydroxyl group of the RNA ribose has never been used to label RNAs in applications of DNA chips or in applications of selective RNA-versus-DNA sorting. The inventors have therefore used this function to react an isatoic anhydride molecule made detectable or functionalised by its conjugation to a fluorophore or to a biotin (or to a molecule of interest). In this way, the RNA will be labelled selectively in the presence of DNA.

By definition, a group of interest is a molecule which:
is a reactive molecule or a reactive group capable of reacting with another reactive molecule or another reactive group in certain conditions (example of a nucleophile and of an electrophile, of an alkyne with an azide, of a maleimide with a thiol, of a diene with an alkene, etc.), and/or
possesses a fluorescence of its own which is different from that of opened isatoic anhydride (anthrilanic ester), and/or
is a ligand recognisable by a recognition molecule or a surface, or a particle, etc., in order to form a stable and possibly reversible complex, of the type biotin/streptavidin, hydrophobic or hydrophilic molecule/hydrophobic or hydrophilic support, antigen/antibody, etc., and/or
is a labelling molecule for a direct reaction chosen from the following molecules:
   an enzyme which produces a signal which is detectable for example by colorimetry, fluorescence, luminescence, such as horseradish peroxidase, alkaline phosphatase, beta-galactosidase, glucose-6-phosphate dehydrogenase,
   chromophores as colorants, or fluorescent, luminescent compounds,
   groups with an electron density detectable by electron microscopy or by their electrical property such as conductivity, amperometry, voltammetry, impedance,
   detectable groups, for example whose molecules are of sufficient size to induce detectable modifications of their physical and/or chemical characteristics, and
   radioactive molecules.
is a labelling molecule for an indirect reaction constituted by a ligand/receptor pair of the type:
   biotin/streptavidin,
   hapten/antibody,
   antigen/antibody,
   peptide/antibody,
   sugar/lectin,
   electrophile molecule/nucleophile molecule,
   polynucleotide/polynucleotide complementary,
   hydrophobic ligand/hydrophobic solid phase, or
   ligand/coordinating metal.

Binding molecule should be understood to mean an isatoic anhydride or derivatives thereof. It should be noted that when isatoic anhydride is opened after reaction with a nucleophile, it then bears the name anthranilate.

Isatoic anhydride derivatives should be understood to mean any organic compounds including a part corresponding to isatoic anhydride and bearing on the aromatic part or on the heterocyclic part of the latter at least one radical, such as a chemical or organic group.

The term functionalisation corresponds to the action of grafting a group of interest onto a nucleic acid by covalent or non-covalent bonding.

Binding arm is defined as an organic spacer arm which can link the binding molecule and the group of interest. It can include a function capable of being cleaved by a physicochemical, photochemical, enzymatic, chemical, thermal means, etc.

Recognition or capture means or molecule is defined as a molecule which may or may not be immobilised on a solid support having a strong affinity for the group of interest.

The definition of an RNA is a natural or synthetic polymer constituted of a least two modified or unmodified successive ribonucleotide units.

The term DNA is defined by a natural or synthetic polymer constituted of a least two modified or unmodified successive deoxyribonucleotide units.

It is also possible to use single chimeric strands constituted of at least one DNA segment and at least one RNA segment.

Inhibition is understood to be the inability of RNA excessively functionalised by an isatoic anhydride derivative to be amplified by a technology for amplifying genetic material (NASBA, PCR, etc.)

By definition, the term sugar means a ribose or deoxyribose compound.

The present invention relates to a process of functionalising at least one ribonucleic acid (RNA) molecule which comprises the following steps:

a) having at least:
a binding molecule constituted by an isatoic anhydride or a derivative thereof,
a group of interest, and
a binding arm linking the binding molecule with the group of interest,
b) reacting the anhydride function of the binding molecule with at least one hydroxyl group in:
   position 2′ of the ribose of one of the RNA nucleotides, and/or
   position(s) 2′ and/or 3′ of the ribose of the nucleotide at the 3′ terminal end of the RNA, and
c) obtaining an anthranilate linking, via the binding arm, the RNA to the group of interest.

According to a first embodiment, the process involves labelling at least one ribonucleic acid (RNA) molecule, which comprises the following steps:
a) having at least:
   a binding molecule constituted by an isatoic anhydride or a derivative thereof, which has an intrinsic fluorescence,
   a group of interest, which has an intrinsic fluorescence signal, but which is different from the signal emitted by the binding molecule, or which does not have an intrinsic fluorescence signal, and
   a binding arm linking the binding molecule with the group of interest,
b) reacting the anhydride function of the binding molecule with at least one hydroxyl group in:
   position 2′ of the ribose of one of the RNA nucleotides, and/or
   positions 2′ and/or 3′ of the ribose of the terminal nucleotide in position 3′ of the RNA, and
c) obtaining an anthranilate linking, via the binding arm, the RNA to the group of interest.

According to a second embodiment, the process involves capturing or separating at least one ribonucleic acid (RNA) molecule, which comprises the following steps:
a) having at least:
   a binding molecule constituted by an isatoic anhydride or a derivative thereof,
   a group of interest constituted by a ligand which is complementary to an anti-ligand, and
   a binding arm linking the binding molecule with the group of interest,
b) reacting the anhydride function of the binding molecule with at least one hydroxyl group in:
   position 2′ of the ribose of one of the RNA nucleotides, and/or
   positions 2′ and/or 3′ of the ribose of the terminal nucleotide in position 3′ of the RNA, and
c) obtaining an anthranilate linking, by means of the binding arm, the RNA to the group of interest,
d) capturing or separating RNA through a ligand—anti-ligand reaction.

Whatever the embodiment of the process and according to one embodiment variant, the binding arm is associated with the binding molecule before said binding arm is associated with the group of interest.

Whatever the embodiment of the process and according to another embodiment variant, the binding arm is associated with the binding molecule after said binding arm is associated with the group of interest.
In the two preceding scenarios, the binding molecule is associated with the RNA beforehand.

The present invention relates to a process of selectively capturing at least one RNA molecule using at least one binding molecule, a group of interest, constituted by a ligand which is complementary to an anti-ligand, and a binding arm linking the binding molecule with the group of interest, the binding molecule being constituted by an isatoic anhydride or a derivative thereof, which attaches via a covalent bond to a hydroxyl group in:
position 2′ of the ribose of one of the RNA nucleotides, and/or
position 2′ and 3′ of the ribose of the terminal nucleotide in position 3′ of the RNA, and/or
position 3′ of the ribose of said terminal nucleotide in position 3′ of the RNA.

The present invention also relates to a process of separating RNA molecules from other biological constituents, in particular DNA molecules, consisting in:
applying the capture process, such as described above, to a biological sample containing undifferentiated nucleic acids (RNA and DNA), the groups of interest being associated with at least one solid support which is easily separable from the rest of the biological sample, and
separating the binding molecules bearing the RNA molecules from the rest of the biological sample.

The present invention relates to another functionalising reagent with formula (I):

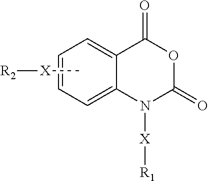

wherein:
R₁ represents H or a group of interest,
R₂ represents H or a group of interest which can be:
   a. a label or a labelling precursor, or
   b. a ligand recognisable by a recognition molecule or a surface, or a particle, etc., in order to form a stable complex,
if R₁ is represented by H, R₂ is represented by a group of interest, and vice-versa, and
X is a binding arm, which links the group of interest to the binding molecule.

If the reagent is a capture or separation reagent, such as described above, the capture or separation means is constituted by a solid support, such as magnetic or non-magnetic polymer or silica particles, or a filter, or even by the inner wall of a receptacle.

If the reagent is a functionalising reagent, such as described above, wherein the binding arm X is:
a single covalent bond linking an atom of the binding molecule and an atom of the group of interest, or
an organic binding arm, such as a single covalent bond between the binding molecule and the group of interest or a single carbon atom, possibly substituted, a chain formation of at least two carbon atoms possibly containing aromatic structures and/or heteroatoms (oxygen, sulfur, nitrogen, etc.).

In this latter embodiment of the functionalising reagent, the binding arm X includes a function or a bond capable of being cleaved by a physicochemical, photochemical, thermal, enzymatic and/or chemical means which separates the binding molecule from the RNA under particular light, temperature, enzymatic or chemical conditions.

The present invention also relates to a functionalised biological RNA molecule capable of being obtained by the process, such as described previously.

The present invention also relates to a kit for detecting a target RNA molecule, comprising a reagent such as described previously.

The present invention finally relates to a functionalising process according to the processes set out above, which comprises the following supplementary step between steps a) and b) consisting of hydrolysing the terminal monophosphate group in position 3′ of each RNA strand to be functionalised.

The invention will be better understood by means of the detailed description which is set out below with regard to the attached figures, namely:

FIG. 1 describes the reaction of isatoic anhydride with a nucleophile.

FIG. 2 shows a labelling reaction of a ribonucleic acid molecule by a binding molecule bearing a group of interest according to the invention.

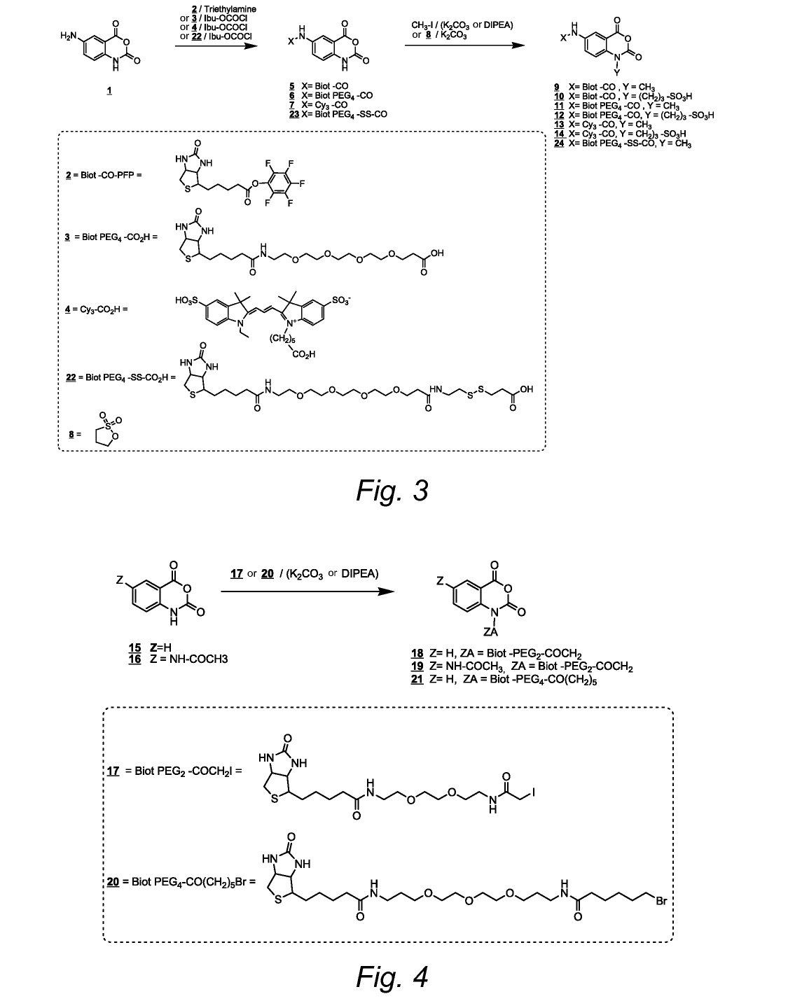

FIG. 3 depicts the synthesis of isatoic anhydride derivatives according to example 1.

FIG. 4 describes the synthesis of isatoic anhydride derivatives according to example 2.

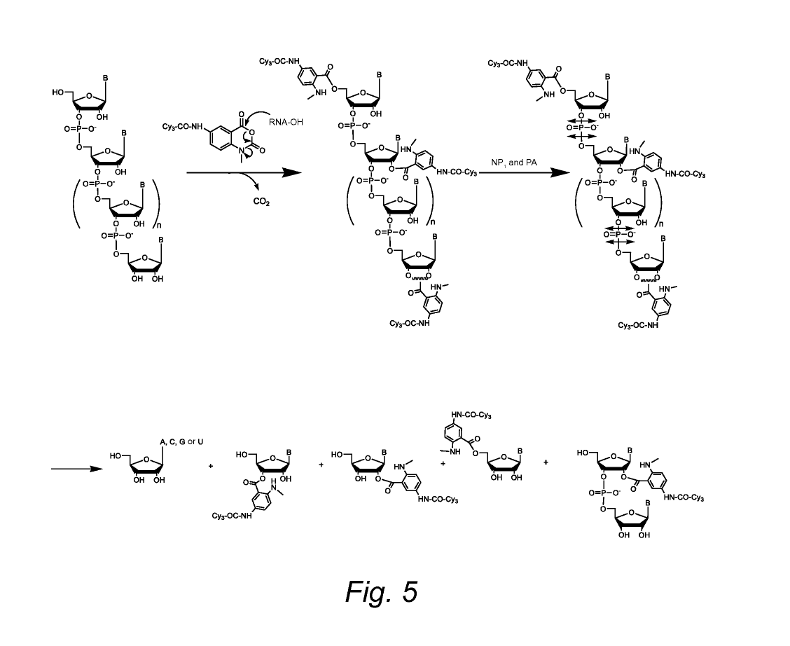

FIG. 5 describes the principle of enzymatic cleaving of an ORN functionalised by Cy3 IA Me (13) and of detecting all possible nucleoside adducts.

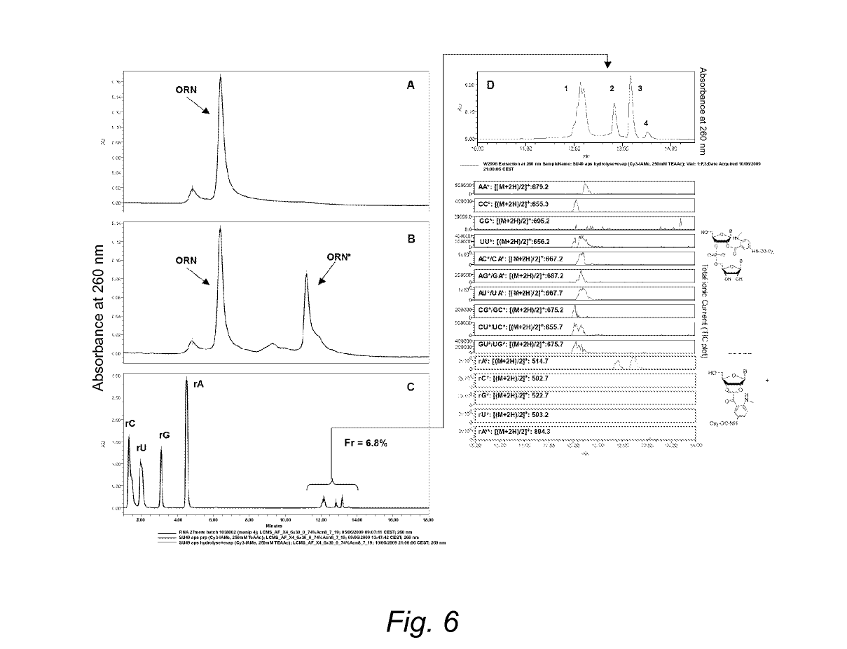

FIG. 6 proposes characterising and identifying by LC-MS the different nucleoside adducts formed during the enzymatic hydrolysis of an ORN labelled by compound 13.

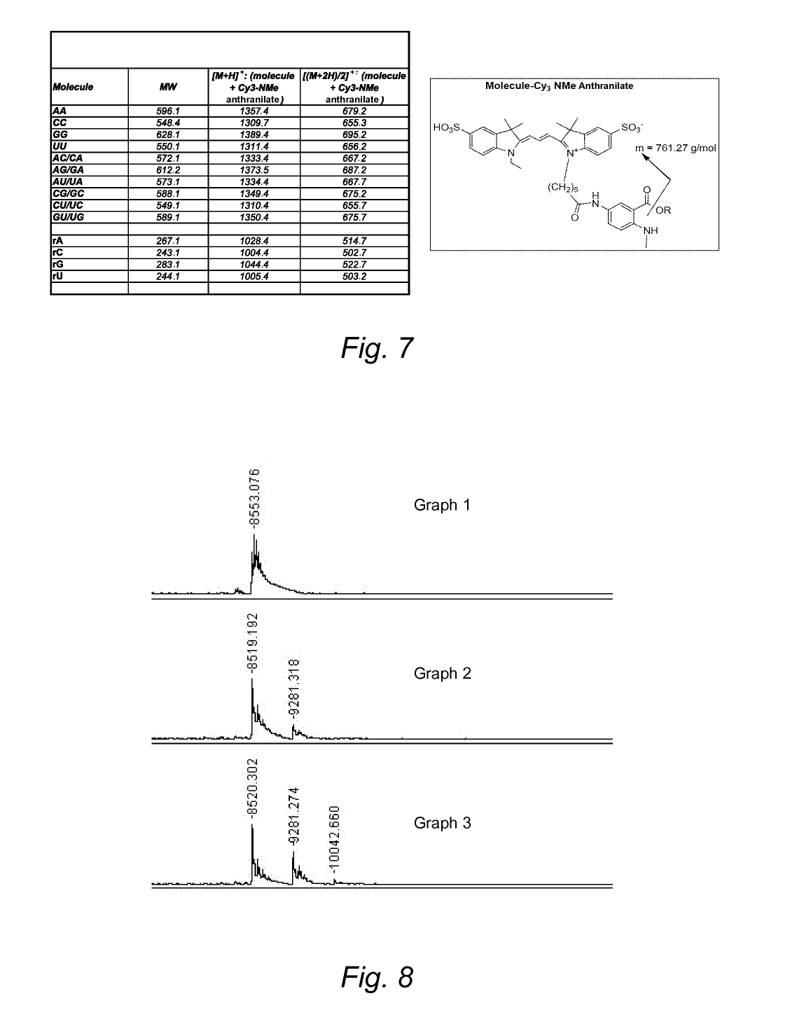

FIG. 7 shows the mass of the acylated or unacylated nucleosides and dinucleotides which can be detected from the sequence described and according to example 3.

FIG. 8 shows the mass spectrograms (Maldi T of) of the functionalisation of an ORN of 27 bases as a function of the modified isatoic anhydride concentration (Cy3 IA Me 13).

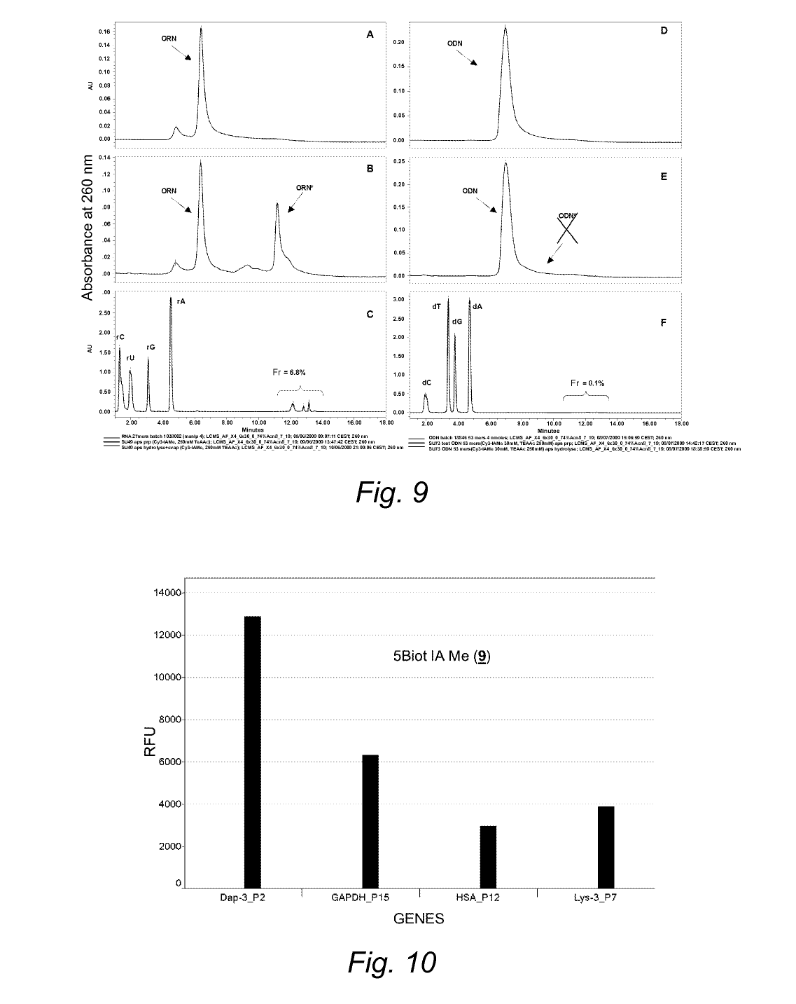

FIG. 9 depicts the reaction monitoring by HPLC between an ODN or an ORN reacted with compound 13.

FIG. 10 depicts the fluorescence intensities (RFU) detected during hybridisation of 4 biotinylated ORNs by compound 9 on an Affymetrix DNA chip.

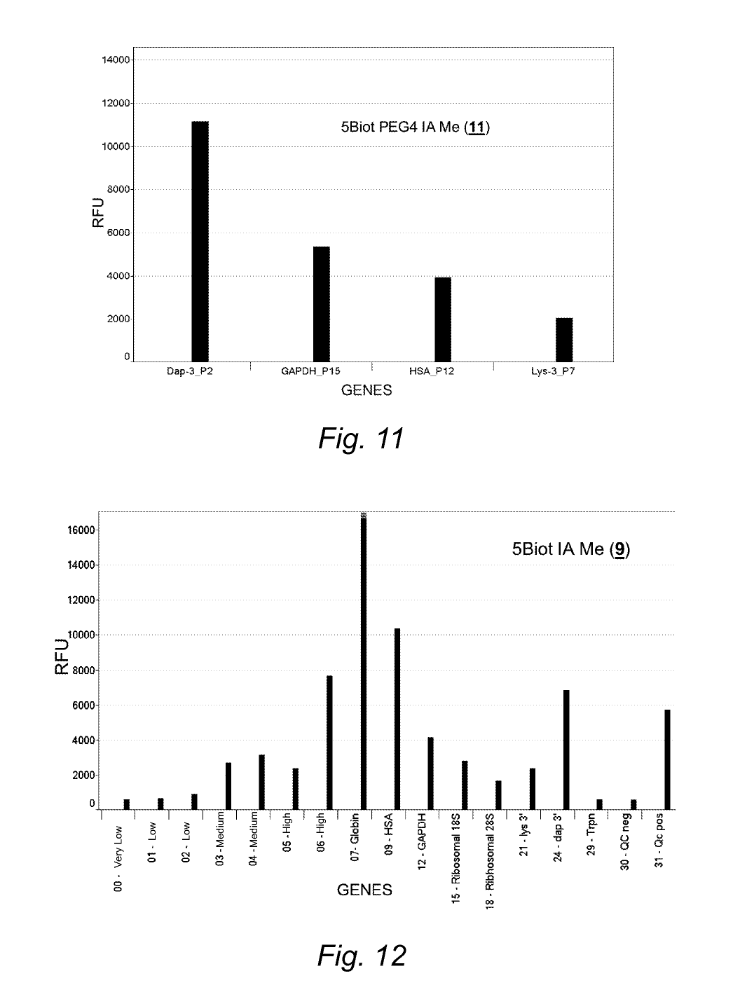

FIG. 11 depicts the fluorescence intensities (RFU) following the detection on a DNA chip of 4 ORN of 60 bases biotinylated with the compound II (Affymetrix Chip).

FIG. 12 depicts the fluorescence intensities (RFU) following the detection on a DNA chip of biotinylated IVTs with compound 9 (Affymetrix Chip).

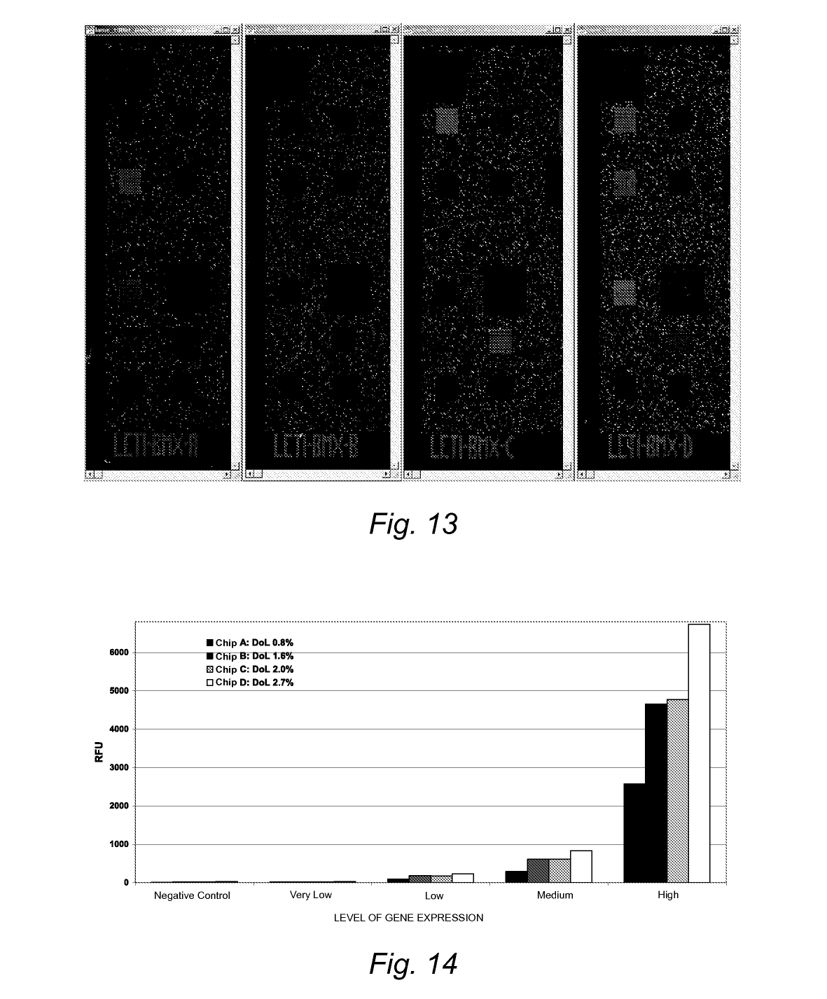

FIG. 13 is a photograph of the DNA chips on AGILENT glass slides, upon detection of the hybridisation of IVTs hybridised and functionalised by the compound Cy3 IA Me 13.

FIG. 14 represents the histogram of the median intensities of the fluorescence detected on an Agilent glass slide chip as a function of the class of the sequences (IVT functionalised with the compound Cy3 IA Me 13).

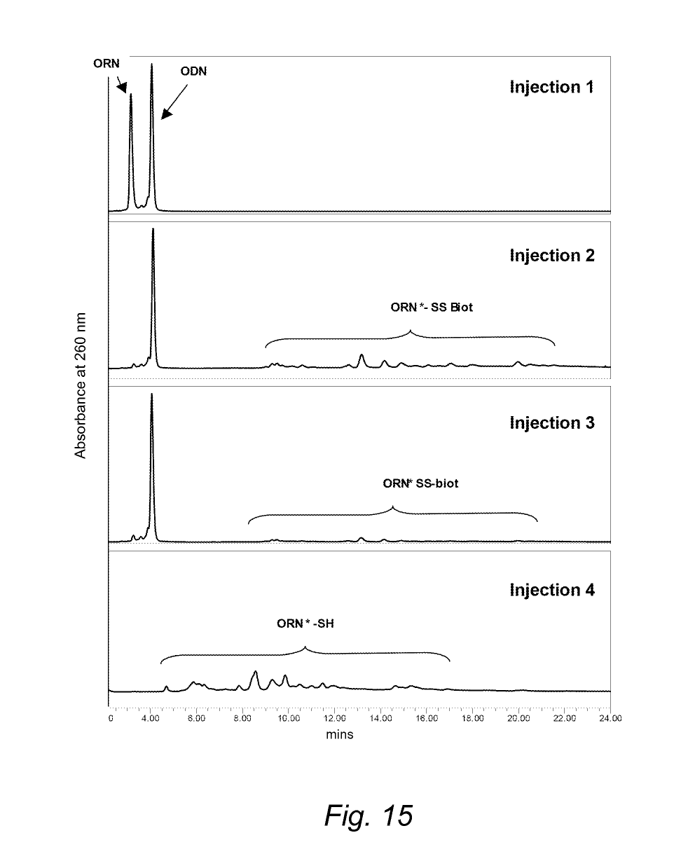

FIG. 15 represents the 260 nm chromatograms of the process of selective functionalisation/capturing and cleaving/elution of RNA, from an ORN/ODN mixture as described in example 11.

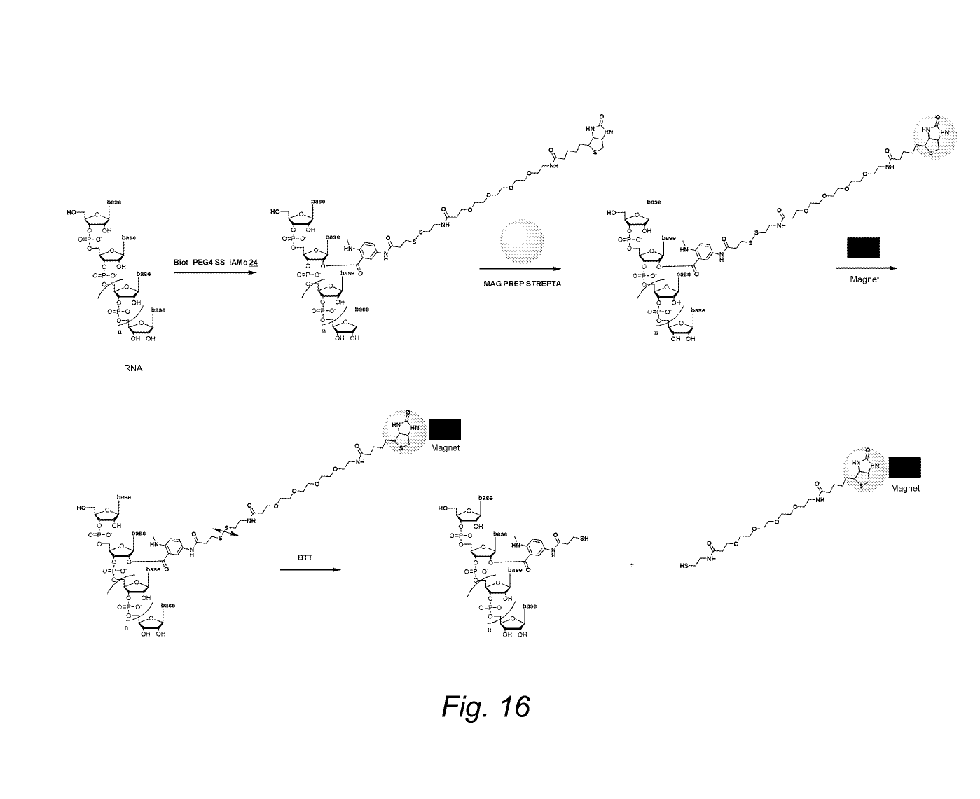

FIG. 16 shows the execution of a protocol of functionalisation, capture, cleaving and elution of an RNA transcript subjected to the action of the compound 5 Biot PEG₄ (SS) IA Me (24).

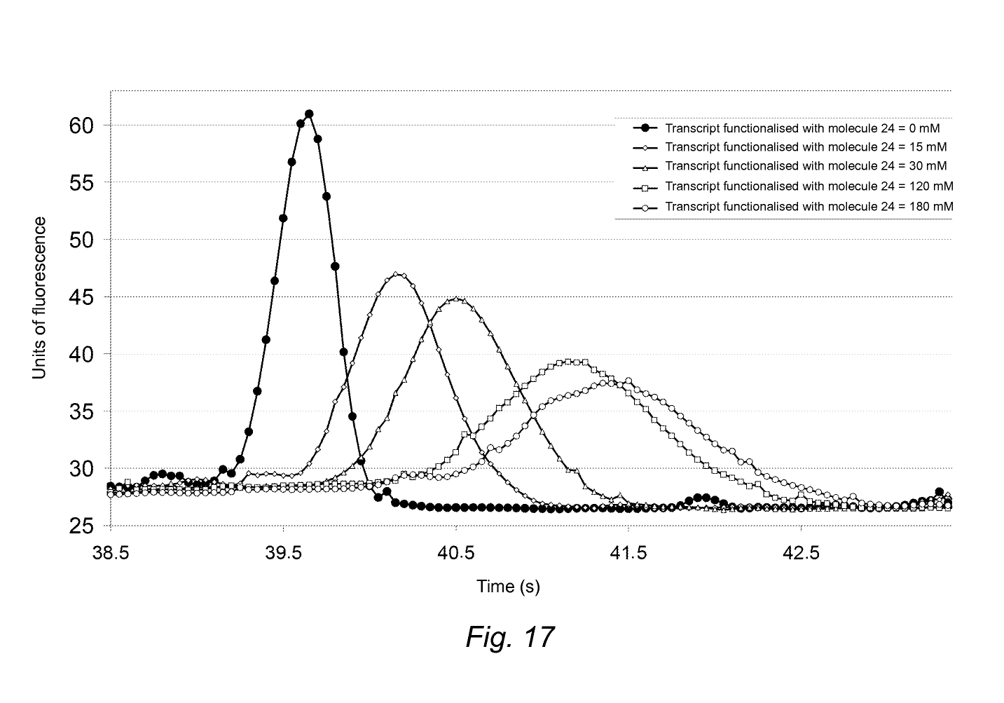

FIG. 17 shows the electrophoretic profile of an HIV transcript which is not functionalised, and functionalised with different concentrations of the molecule 5 Biot PEG₄ (SS) IA Me (24).

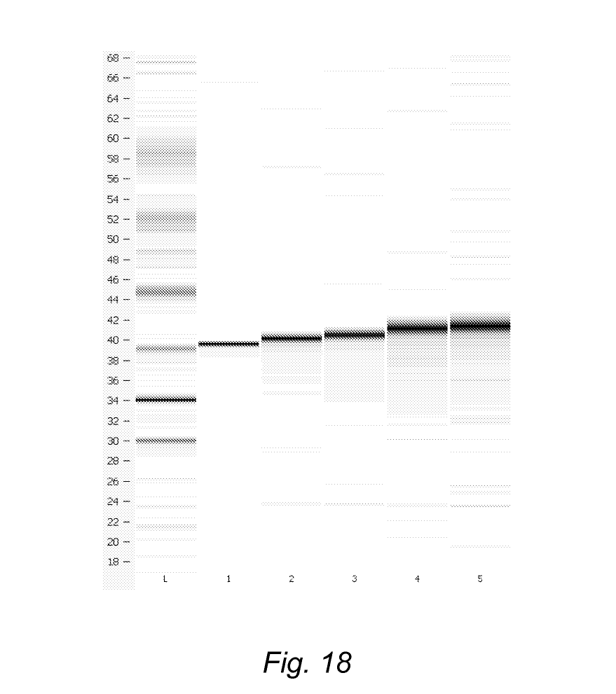

FIG. 18 shows the same thing as FIG. 17 but in another graphic representation (gel type).

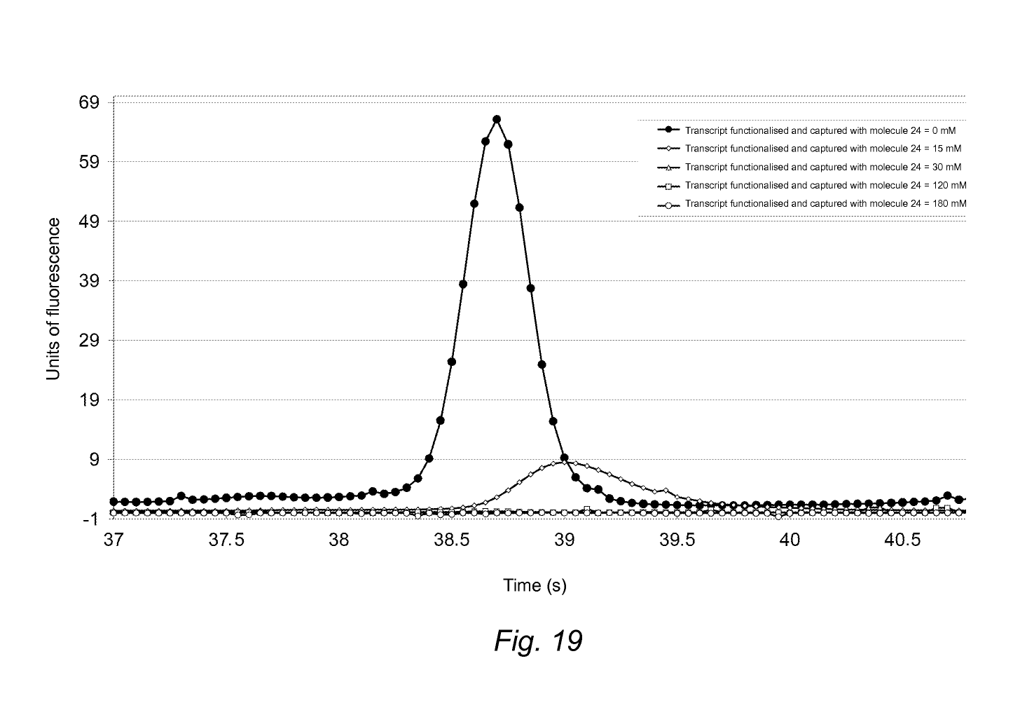

FIG. 19 shows the electrophoretic profile corresponding to the supernatant of an HIV transcript which is not functionalised, and functionalised with different concentrations of the molecule 5 Biot PEG₄ (SS) IA Me (24) exposed to streptavidin-coated magnetic particles.

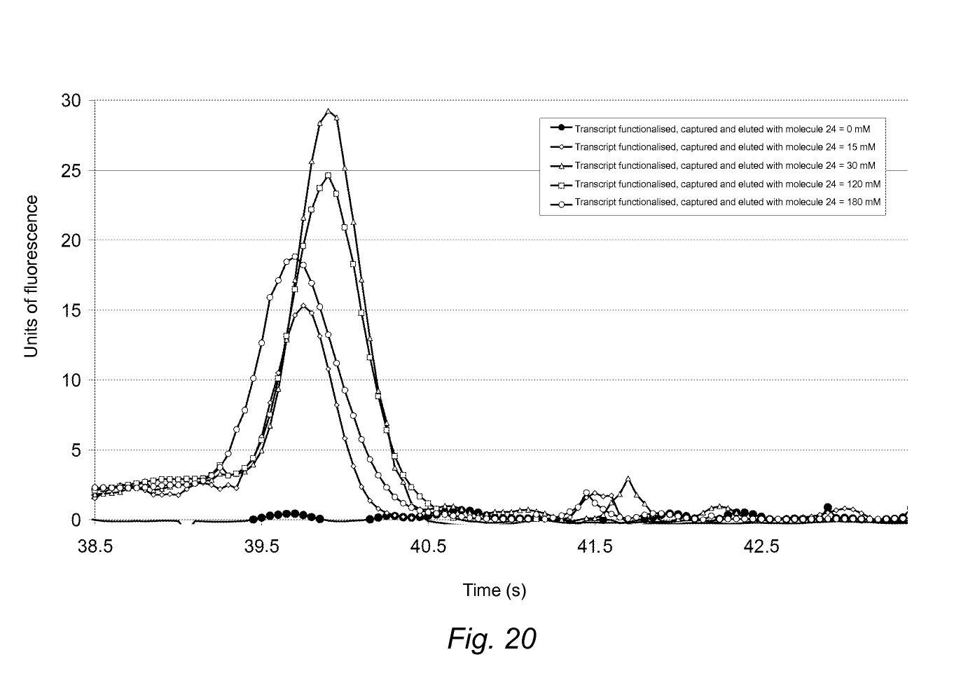

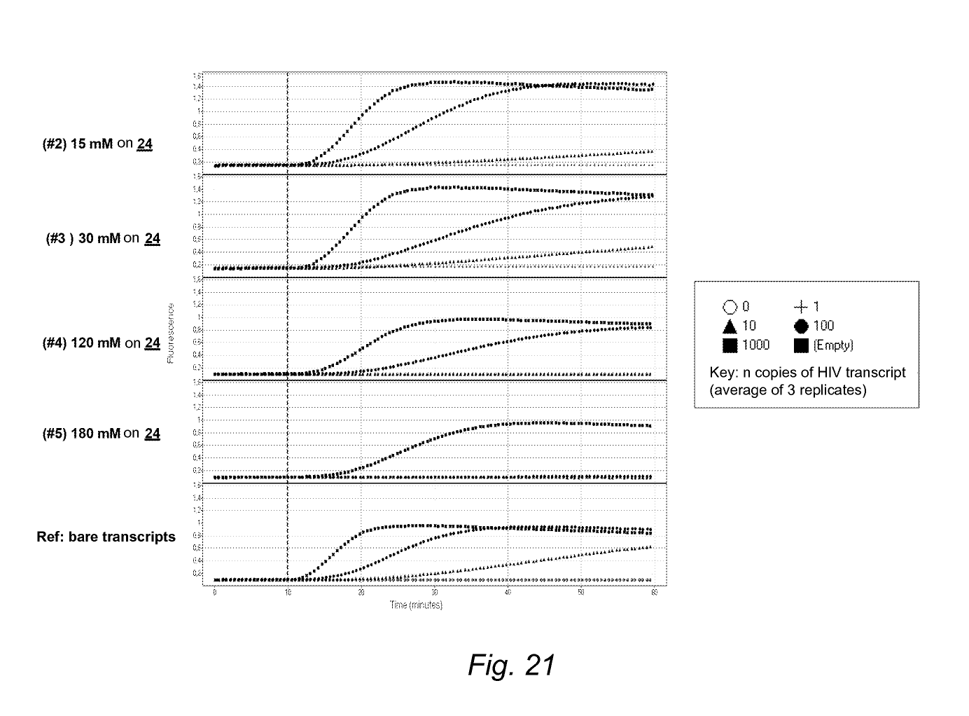

FIG. 20 shows the electrophoretic profile of an HIV transcript which is not functionalised and functionalised with different concentrations of the molecule 5 Biot PEG₄ (SS) IA Me (24) exposed to streptavidin-coated magnetic particles, then eluted by chemically cleaving of the disulfide bond (SS).

FIG. 21 represents a superposition of ranges of HIV transcripts (1000, 100, 50, 10 and 1 copy(copies)) functionalised with the molecule (24) at different concentrations (15, 30, 120 and 180 mM), captured on streptavidin-coated magnetic particles, then eluted by chemical cleaving with DTT and amplified by NASBA.

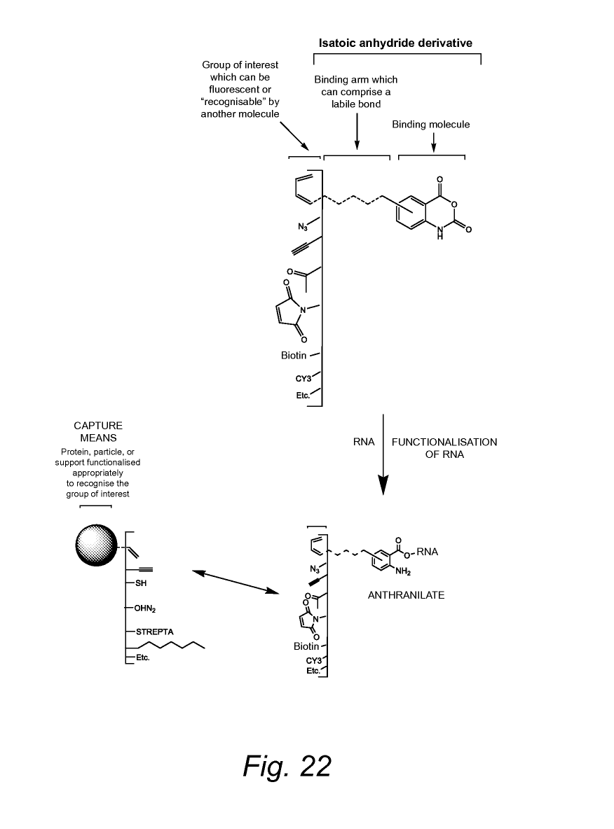

Finally, FIG. 22 represents the reaction diagram of the functionalisation of RNA according to the present invention by means of a particular isatoic anhydride.

The following abbreviations will be used in the examples described below:
ACN: Acetonitrile,
Ar: aromatic,
TLC: thin-layer chromatography,
CDCl₃: Deuterated chloroform,
d: doublet,
DCM: dichloromethane,
dd: split doublet,
DMF: dimethylformamide,
DMSO-d6: dimethyl sulfoxide,
DMSO-d6: deuterated dimethyl sulfoxide,
MilliQ water: Ultrapure water (Millipore, Molsheim, France),
Eq: equivalents,
HPLC: high-performance liquid chromatography,
IA: isatoic anhydride,
IVT: in vitro transcripts
M: multiplet,
m: cluster,
Me: methyl,
MeOH: methanol,
Nb exp: Experiment repetition number,
nd: not determined,
NMO: N-methylmorpholine,
NP1: Nuclease P1,
ODN: Oligo-deoxyribonucleotide,
ORN: Oligo-ribonucleotide,
AP: Alkaline phosphatase,
PBS 1x: Phosphate Buffered Saline=(0.01 M PO₄⁻, 0.0027 M KCl, 0.137 M NaCl, pH=7.4 at 25° C. SIGMA ref. 4417, Saint Louis, USA),
q: quadruplet,
Yld: yield,
Rf or RT: retention time,
NMR: nuclear magnetic resonance,
rpm: revolutions per minute,
s: singlet,
SS: disulfide bond,
t: triplet,
TEAAc: Triethyl ammonium acetate,
Fr: functionalisation rate,
AT: Ambient temperature,
UV: ultraviolet.

### The General Conditions for the Analysis and the Synthesis of the Chemical Compounds Used in the Following Examples are Described Below:

The LC-MS analyses were carried out with a HPLC WATERS Alliance 2795 chain equipped with a PDA 996 (Waters) diode array detector, a ZQ 2000 (Waters) mass spectrometry detector, Empower version 2 software and a WATERS XTerra MS C18 column (4.6×30 2.5 μM) used with a flow of 1 ml/minute at 30° C. (detection at 260 nm or in max plot). The ZQ 2000 mass spectrometer possesses an Electrospray ionisation source. The ionisations were performed in positive mode with a cone voltage of 20 V and a capillary voltage of 3.5 kV.

Several Types of Gradients have been Used for the HPLC Analyses

| | | | Eluent C | |
|---|---|---|---|---|
| | | | (Ammonium | |
| Conditions | Eluent A | Eluent B | formate AF) | Linear gradient |
| 1 | MilliQ | ACN | AF 500 mm | 97% of A/1% of |
| | water | | pH 7 | B to 62% of A/ |
| | | | | 36% of B in 10 |
| | | | | mins with 2% of |
| | | | | eluent C in |
| | | | | isocratic |
| 2 | MilliQ | ACN | AF 500 mm | 97% of A/1% of |
| | water | | pH 7 | B to 34% of A/ |
| | | | | 64% of B in 18 |
| | | | | mins with 2% of |
| | | | | eluent C in |
| | | | | isocratic |
| 3 | MilliQ | ACN | AF 500 mm | 98% of A/0% of |
| | water | | pH 7 | B to 24% of A/ |
| | | | | 74% of B in 18 |
| | | | | mins with 2% of |
| | | | | eluent C in |
| | | | | isocratic |
| 4 | TEAAC | ACN/Water | — | 86% of A/14% of |
| | 50 mM | 50/50 | | B to 45.5% of A/ |
| | | with 50 | | 54.5% of B in |
| | | mM of | | 23 minutes |
| | | TEAAc | | |

The NMR spectra were recorded on a BriAker 200 or 500 MHz spectrometer (only for molecule 13). The chemical shifts (5) are given in ppm relative to the solvent peak taken as an internal reference (CDCl₃: 7.24 ppm; DMSO-d₆: 2.49 ppm; D₂O: 4.80 ppm at 25° C.). The spectra are described with the abbreviations above: s, d, t, q, qu, m and M. The coupling constants (J) are expressed in Hertz (Hz)

The absorbance spectra were recorded on a UV-Visible Varian spectrophotometer, Cary 300 Bio model. The analyses by fluorescence were carried out on a Varian fluorimeter, Cary Eclipse model (Varian, Santa Clara, Calif., USA).

The ionic chromatography analyses were performed on a DIONEX ICS 1000 chromatograph (Sunnyvale, Calif., USA)

in cation mode on an IonPac CS12A column (Sunnyvale, Calif., USA).

The thin layer chromatography analyses were performed on ALUGRAM® MACHEREY-NAGEL SIL G/UV₂₅₄ silica plates, 4×8 cm, (Duren, Germany) with UV detection at 254 nm or reverse phase TLC (Macherey Nagel, Duren, Germany, Alugram RP-18W 40×80 mm).

The purification of the products was performed by chromatography on FLUKA Silica gel 60 (40-63 μm). The conditions of separation by “flash” chromatography (under argon pressure) strictly respect the conditions described by Clark Still et al. (Clark Still, W.; Kahn, M.; Mitra, A. J. Org. Chem. 1978, 43, 2923-2925) i.e. fixed height of silica of 15 cm driven at a flow of 5 cm/minute, with the diameter of the column depending on the quantity and the Rf of the products to be purified. The purification of certain hydrophilic products was performed by chromatography on silica gel, MERCK RP-18 silica gel (40-63 μm). The analyses by thin layer chromatography were performed on MERCK 18 F₂₅₄ silica plates and viewed under UV at 254 nm or directly by the naked eye.

### PREAMBLE TO EXAMPLES 1 AND 2

General Description of the Synthesis of the Compounds which Will be Described in Examples 1 and 2

The conjugation of isatoic anhydride or its derivatives to a group of interest implies a chemical reaction between the isatoic anhydride, which possesses a reactive function, and the molecule of interest, which also possesses a reactive function. It should be noted that it is particularly important to preserve the integrity of the isatoic anhydride part during this coupling. The person skilled in the art knows a multitude of ways of thus conjugating two molecules together in order to obtain a new molecule which has properties common to both.

### EXAMPLE 1

### Synthesis of Alkylated Derivatives of Isatoic Anhydride which are Conjugated to a Group of Interest Borne by the Aromatic Ring

In a particular embodiment of the invention, isatoic anhydride is conjugated to a group of interest, for example a label such as Cy3, biotin, biotin possessing a labile group, which is coupled to the binding molecule which is isatoic anhydride via an aromatic ring, as is clearly shown in FIG. 3.

We have thus chosen to link the isatoic anhydride and the molecule of interest via the aromatic part of the isatoic anhydride, with an amide-type binding arm. This is done by reacting (method 1):
5-amino IA (1) (Sigma-Aldrich, Saint Louis, USA)
with a carboxylic acid (biotin, with or without a hydrophilic arm (3) or chemolabile arm (22)), or Cy3-COOH (4))
with a coupling reagent (isobutyl chloroformate).
So as to obtain compounds (6), (23) or (7).

Since the 5-amino group of the isatoic anhydride is not very nucleophilic, we have preferred to use method 1 rather than perform the peptide coupling directly by reaction between (method 2):
5-amino IA (1)
and an activated ester of the molecule of interest.

Method 1 makes it possible to access the conjugates of isatoic anhydride (Biot PEG4 IA (6), Cy3 IA (7) or Biot PEG4 SS IA (23) with a far better yield than the direct coupling of 5-amino IA with biotin PFP (2) such as we did to access the compound Biot IA (5) (method 2).

The biotinylated derivative (3) was chosen because it is a hydrophilic compound which is easily detectable with a fluorescent streptavidin molecule.

The biotinylated derivative (22) was chosen because it is a hydrophilic compound which is easily detectable with a streptavidin molecule, and furthermore possesses chemolabile disulfide bond (SS) which makes it possible to free, in the medium, the RNA molecule which was captured.

Cyanine 3 Cy3-COOH (4) was chosen as an example of a group of interest because it is a fluorophore commonly used in biomedical applications or molecular diagnosis applications due to its photostability and its fluorescence properties at wavelengths far removed from the autofluorescence of certain biological constituents ((λ_{excitation}=545 nm and λₑₘᵢₛₛᵢₒₙ=570 nm). This molecule was synthesised by following the synthesis route described by Waggoner et al. (Waggoner A. S., Mujumdar R. B., Ernst L. A., Mujumdar S. R., Lewis C. J. Cyanine Dye Labeling Reagents: Sulfoindocyanine Succinimidyl Esters, Bioconjugate Chemistry, 1993, 4, 105-11).

It should be noted that it is understood that the person skilled in the art would know to use other conventional organic chemistry reactions to couple isatoic anhydride to any other molecule of interest via bonds and groups situated at various positions on the aromatic ring. It is therefore also understood that we do not limit ourselves to position 5, and that other substitution positions may be used and even act to modulate the reactivity of isatoic anhydride to nucleophiles.

In order to increase the reactivity of these compounds to 2′OHs of the RNA, we introduced various groups onto the intracyclic —NH— of the isatoic anhydride by alkylation with halogen compounds or compounds which are reactive to this function, which increases the reactivity, following an alkylation of this position. The compounds 5Biot IA Me (9), 5Biot PEG4 Me (11), 5Biot PEG4 SS IA Me (24) or Cy3-IA Me (13) were thus obtained by reaction of the corresponding precursor compounds with methyl iodide. It should be noted that other alkylating compounds may be used so as to increase the reactivity of the isatoic anhydride to nucleophilic groups (hydroxyls of the RNA in this case), but also to provide a supplementary functionality (solubility, stability of the duplex, labelling, etc.) whilst retaining good reactivity to nucleophiles. The compounds 5Biot IA S03-10, 5Biot PEG4 503-12 or Cy3-IA S03-14 were thus obtained by reaction of the corresponding precursor compounds with sultone 8.

It is understood that a very large number of electrophilic compounds may thus be reacted with the NH group of the isatoic anhydride or its derivatives so as to modulate the properties thereof. It is understood that a large number of reactions and reagents make it possible to alkylate the derivatives of isatoic anhydride on the NH.

### EXAMPLE 1.1

### Synthesis of Biotin and Pentafluorophenol Ester (2)

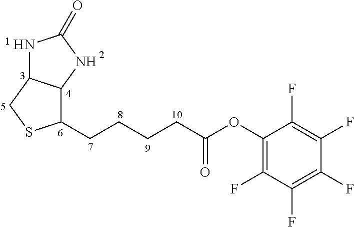 [SMILES: O=C1NC2CSC(CCCCC(=O)Oc3c(F)c(F)c(F)c(F)c3F)C2N1]

Biotin (5 g; 23.1 mmol; 1.0 eq) is suspended in anhydrous DMF (50 mL) and pyridine (2.07 mL; 25.4 mmol; 1.1 eq). After 5 minutes of stirring, pentafluorophenyl trifluoroacetate (PFP-TFA: 4.621 mL, i.e. 7.50 g; 25.4 mmol; 1.1 eq) is added. The reaction is terminated after one night of stirring. The solvents are evaporated with a rotary evaporator. The evaporation residue is taken up with 100 mL of ethyl ether to be suspended, then spin-dried on sintered glass. The residue is rinsed with a minimum of ether.

Mass obtained=7.106 g, i.e. a yield of 81%. Normal-phase TLC eluents: DCM/MeOH: 90/10.

M+H, 411.1 g·mol⁻¹

NMR 1H (200 MHz, DMSO): 1.2 to 1.8 (m, 6H, 7-8-9); 2.3 to 2.9 (m, 4H, 5-10); 3.05 (q, 1H, 6); 4.00 and 4.40 (M, 2H, 3-4); 6.2 and 6.5 (s, 2H, 1-2).

### EXAMPLE 1.2

### Synthesis of 5-biotin-isatoic anhydride (5)

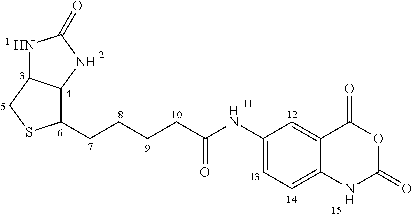 [SMILES: O=C(CCCCC1SCC2NC(=O)NC21)Nc1ccc2[nH]c(=O)oc(=O)c2c1]

5-amino-isatoic anhydride 1 (500 mg; 2.81 mmol; 1.0 eq) is placed in solution in a mixture of DMF (14 mL) and triethylamine (3.15 mL; 22.4 mmol; 8.0 eq). The solution obtained, dark brown in colour, is stirred for 5 mins at AT before simultaneously adding the biotin and pentafluorophenol ester (2) (1.6125 g; 3.93 mmol; 1.4 eq). The reaction is left under stirring for 16 h. The solvents are evaporated until dry, then co-evaporated twice with anhydrous acetonitrile with a rotary evaporator. The evaporation residue is taken up in DMF (21.2 mL) and triethylamine (4.80 mL; 33.92 mmol; 8.0 eq) then stirred for 16 h. Hydrochloric acid (38 mL; 1 M; 15.8 eq) at 2° C. is added to the mixture before being stirred then centrifuged at 7000 rpm for 5 mins. The supernatant is eliminated and the pellet is taken up three times in succession in water (68 mL), stirred, centrifuged and the supernatant is eliminated again. The pH of the supernatant must be equal to 7. The pellet is then dried several times in succession with ethyl ether in order to facilitate the drying in the oven under vacuum at ambient temperature. The product is taken up in a minimum of DMF to which one spatula of RP18 silica is added, this mixture is evaporated and then co-evaporated twice with acetonitrile before being deposited onto a reversed-phase chromatographic column of 3 cm in diameter by 10 cm in height (Merck LiChroprep RP-18 silica (40-63 μm) ref.: 1.13900.1000). The products are eluted with a mixture, acetonitrile/DMF:90/10.

Mass obtained=557 mg, i.e. a yield of 32%.

M+H, 405.1 g·mol⁻¹

NMR 1H (200 MHz, DMSO): 1.3 to 1.8 (m, 6H, 7-8-9); 2.34 (t, 2H, 10); 2.6 (d, 2H, 5); 2.9 (dd, 1H, 6); 3.15 (M, 1H, 10); 4.10 and 4.40 (M, 2H, 3-4); 6.47 and 6.39 (s, 2H, 1-2); 7.13 (d, 1H, 14); 7.85 (dd, 1H, 13); 8.30 (s, 1H, 12).

### EXAMPLE 1.3

### Synthesis of 1-Methyl-5 biotin-isatoic anhydride (9)

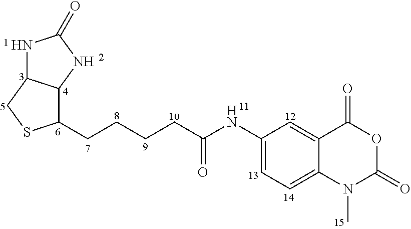 [SMILES: Cn1c(=O)oc(=O)c2cc(NC(=O)CCCCC3SCC4NC(=O)NC43)ccc21]

The product (5) (522 mg; 1.29 mmol; 1.0 eq) is dissolved in DMF (10 mL). Potassium carbonate (375 mg; 2.71 mmol; 2.1 eq) is suspended in this mixture and stirred for 5 minutes at ambient temperature before methyl iodide (105 μl; 1.68 mmol; 1.3 eq) is added. After 1 h20 of stirring, the reaction medium is filtered on sintered glass of porosity 3. A spatula of RP18 silica is added to the filtrate before it is evaporated until dry then co-evaporated 2 times with acetonitrile. The powder obtained is deposited onto a reversed-phase chromatographic column with a diameter of 3 cm and a height of 10 cm (Merck LiChroprep RP-18 silica (40-63 μm) ref.: 1.13900.1000). The products are eluted with two mixtures, firstly water/ACN/DMF: 79/20/1, then water/ACN/DMF:69/30/1. The product (9) is recovered with the second eluent.

Mass obtained=65 mg, i.e. a yield of 12%.

M+H, 419.1 g·mol⁻¹

NMR 1H (200 MHz, DMF): 1.6 to 2.1 (m, 6H, 7-8-9); 2.7 (t, 2H, 10); 2.9 to 3.3 (m, 6H, 5-6-15); 3.5 (M, 1H, 10); 4.5 and 4.8 (M, 2H, 3-4); 6.5 and 6.6 (s, 2H, 1-2); 7.7 (d, 1H, 14); 8.2 (d, 1H, 13); 8.8 (s, 1H, 12)

### EXAMPLE 1.4

### Synthesis of 1-propylsulfone-5-biotin-isatoic anhydride (10)

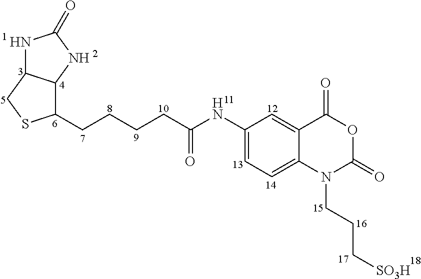 [SMILES: O=C(CCCCC1SCC2NC(=O)NC21)Nc1ccc2c(c1)c(=O)oc(=O)n2CCCS(=O)(=O)O]

The product (5) (50 mg; 123.6 μmol; 1.0 eq) is dissolved in DMF (1.2 mL; 100 mM). Potassium carbonate (63.6 mg; 408 pmol; 3.3 eq) is suspended in this mixture. After two minutes of stirring in the vortex at 1000 revolutions per minute, the device is placed in a cold chamber at +4° C., before adding propane sultone (142 μl; 161 μmol; 1.3 eq). After 22 hours of stirring, the reaction medium is centrifuged and the supernatant is recovered. The pellet is washed again twice with DMF. A spatula of RP18 silica is added to the supernatant before it is evaporated until dry then co-evaporated 2 times with acetonitrile. The powder obtained is deposited onto a reversed-phase chromatographic column with a diameter of 1 cm and a height of 10 cm (Merck LiChroprep RP-18 silica (40-63 μm) ref.: 1.13900.1000). The products are eluted with two mixtures, firstly ACN/DMF:100/0, then ACN/DMF:90/10, ACN/DMF:80/20, ACN/DMF:60/40, ACN/DMF:0/100. The product (10) is recovered with the ACN/DMF eluent: 90/10.

Mass obtained=23 mg, i.e. a yield of 35%.

M+H, 527.1 g·mol⁻¹

NMR 1H (200 MHz, DMSO): 1.3 to 1.75 (m, 6H, 7-8-9); 1.90 (qu, 2H, 16); 2.31 (t, 2H, 10); 2.48 (M, 2H, 15); 2.65 (M, 2H, 5); 2.85 (dd, 1H, 6); 3.15 (M, 1H, 10); 4.00 and 4.40 (M, 2H, 3-4); 6.47 and 6.38 (s, 2H, 1-2); 7.64 (d, 1H, 14); 7.90 (dd, 1H, 13); 8.42 (sd, 1H, 12); 10.22 (s, 1H, 18)

### EXAMPLE 1.5

### Synthesis of 5-biotin-hexyl ethylene glycol-isatoic anhydride (6)

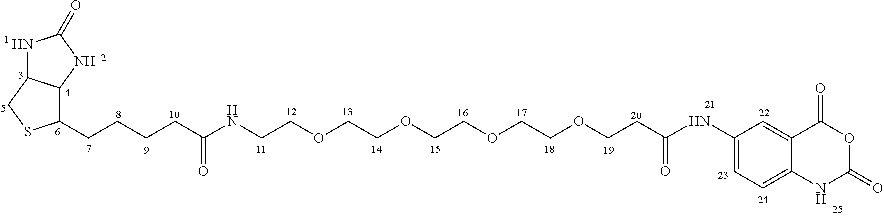 [SMILES: O=C(CCCCC1SCC2NC(=O)NC21)NCCOCCOCCOCCOCCC(=O)Nc1ccc2[nH]c(=O)oc(=O)c2c1]

5-amino-isatoic anhydride (1) (663 mg; 3.72 mmol; 1.0 eq) is dissolved in DMF (18.6 mL; 200 mM) dried and co-evaporated twice in acetonitrile before being finally taken up in 18.6 mL of DMF. Then N-methylmorpholine NMO (409 μl; 3.72 μmol; 1.0 eq) and isobutyl chloroformate (482 μl; 3.72 μmol; 1.0 eq) are added to the medium after cooling to 0° C. in an ice bath. After minutes, Biot-EG4-COOH (3) (1.831 g; 3.72 mmol; 1.0 eq, QuantaBio design, Powell USA, ref.: 10199) is added in powder form. In order to increase the yield of the reaction, successive additions of reagents are added over time as summarised in table 1 below.

TABLE 1
details of the successive additions of reagents
to the reaction for accessing compound 6

| | | | Isobutyl | |
|---|---|---|---|---|
| | Compound (1) | NMO | chloroformate | Compound (3) |
| T = 0 min | 0.663 g/1.0 eq | 0.409 mL/1.0 eq | 0.482 mL/1.0 eq | 1.831 g/1 eq |
| T = 120 min | 1.326 g/2.0 eq | 0.818 mL/2.0 eq | 0.964 mL/2.0 eq | 1.831 g/1 eq |
| T = 165 min | 1.642 g/2.5 eq | 1.022 mL/2.5 eq | 1.205 mL/2.5 eq | 1.831 g/1 eq |
| T = 205 min | 1.899 g/3.0 eq | 1.227 mL/3.0 eq | 1.446 mL/3.0 eq | 1.831 g/1 eq |

In a first stage, the reaction medium is triturated 5 times in acetone and 2 times in ether, in order to obtain a chestnut-brown powder. In a second stage, the powder obtained is deposited onto a reversed-phase chromatographic column with a diameter of 4 cm and a height of 11 cm, (Merck LiChroprep RP-silica (40-63 μm) ref.: 1.13900.1000). The products are eluted with a mixture, water/DMF:50/50. In a last stage, the eluents of interest are evaporated and triturated in ethyl ether.

Mass obtained=447 mg, i.e. a yield of 18%.

M+H, 652.3 g·mol⁻¹

NMR 1H (200 MHz, DMSO): 1.1 to 1.8 (m, 6H, 7-8-9); 2.07 (t, 2H, 10); 2.5 to 3 (m, 7H, 5-6-11-20); 3 to 3.6 (m, 16H, 12 to 19); 4.1 and 4.3 (M, 2H, 3-4); 6.4 and 6.4 (s, 2H, 1-2); 7.1 (d, 1H, 24); 7.8 (dd, 1H, 23); 8.3 (sd, 1H, 22); 10.2 (s, 1H, 21); 11.7 (s, 1H, 25)

### EXAMPLE 1.6

### Synthesis of 1-methyl-5-biotin-hexyl ethylene glycol-isatoic anhydride (11)

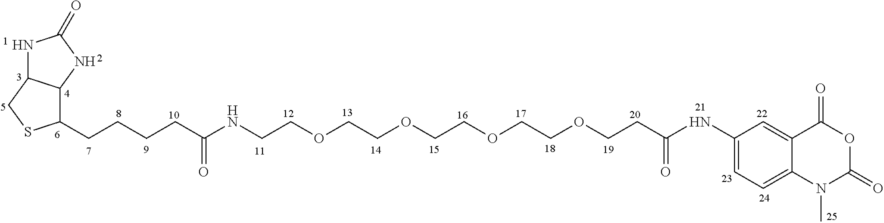 [SMILES: Cn1c(=O)oc(=O)c2cc(NC(=O)CCOCCOCCOCCOCCNC(=O)CCCCC3SCC4NC(=O)NC43)ccc21]

Product 6 (200 mg; 307 μmol; 1.0 eq) is dissolved in DMF (3.05 mL) to which potassium carbonate (89.1 mg; 644 μmol; 2.1 eq) is added. The heterogeneous medium is stirred for 5 minutes at ambient temperature before adding methyl iodide (25 μl; 400 μmol; 1.3 eq). After 90 minutes of stirring, the reaction is filtered on filter paper, then the filtrate is evaporated until dry before being solubilised in 500 μl of DMF. The solution is deposited on a reverse-phase chromatographic column with a diameter of 2 cm and a height of 10 cm. The product is eluted with ACN: 100%. The fractions are evaporated until dry, then the solid obtained is triturated in ethyl ether.

Mass obtained=240.4 mg, i.e. a yield of 100%.

M+H, 666.3 g·mol⁻¹

NMR 1H (200 MHz, DMSO): 1.1 to 1.7 (m, 6H, 7-8-9); 2.05 (t, 2H, 10); 2.5 to 3 (m, 7H, 5-6-11-20); 3 to 3.7 (m, 16H, 12 to 19); 3.9 (M, 3H, 25); 4.1 and 4.3 (M, 2H, 3-4); 6.38 and 6.44 (s, 2H, 1-2); 7.4 (d, 1H, 24); 7.9 (dd, 1H, 23); 8.4 (sd, 1H, 22); 10.2 (s, 1H, 21)

### EXAMPLE 1.7

### Synthesis of 5-Cy3 isatoic anhydride (7)

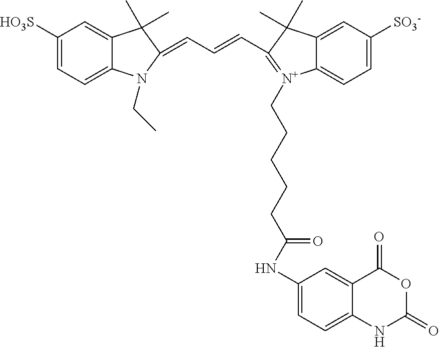

In a 10 mL round-bottomed flask under argon at 0° C. (ice bath), 249 mg of Cy3-COOH 4 (0.8 mmoles; 1 eq) are placed in solution in 3 mL of DMF. 87 μL of N-methyl morpholine (0.794 mmoles; 1 eq) as well as 104 μL of isobutyl chloroformate (0.798 mmoles; 1 eq) are then added, and the reaction is left under stirring at 0° C. After 15 mins, 213.1 mg of 5-amino isatoic anhydride (1.196 mmoles; 1.5 eq) are added and the reaction medium is left under stirring at ambient temperature. LC-MS monitoring (conditions 1) shows that the reaction is instantaneous. After 20 minutes of reaction, 1 eq of N-methyl morpholine/isobutyl chloroformate are then added, and the reaction is left under stirring for 10 minutes in order to transform the totality of the 5-amino isatoic anhydride into carbamate. The reaction medium is then evaporated, taken up in ethyl ether, filtered on sintered glass and finally washed three times with 15 mL of acetone in order to eliminate the excess isatoic anhydride transformed into carbamate. The pink solid thus obtained is then purified on a C18 silica gel column by using an eluent gradient (H₂O/DMF 75:25, then H₂O then H₂O/DMF 50:50), and then dried under vacuum in order to eliminate the residual DMF. The final product is obtained in the form of a pink powder with a yield of 50% (339 mg; 0.4 mmoles).

M+H=791.9 g·mol⁻¹, TLC: (H₂O/DMF 8/2; UV development) Rf=0.5

LC/MS: condition 1: RT=7.4 min; max absorption λ=266.4; 518.4 and 552.5 nm; [M+H]⁺: 791.5.

¹H NMR (500 MHz, DMSO-d6): δ 1.31 (t, 3H, β′-CH₃, J_{β′-CH3-α′CH2}=7 Hz); 1.44 (m, 2H, γ-CH₂); 1.66 (m, 2H, δ-CH₂); 1.70 (s, 12H, 2×C(CH₃)₂); 1.77 (m, 2H, β-CH₂); 2.32 (t, 2H, ε-CH₂, J_{ε-CH2}-_{δCH2}=7 Hz); 3.98 (m, 2H,); 4.15 (m, 4H, α-CH₂ and α′-CH₂); 6.52 (2d, 2H, vinyl α-CH and vinyl α′-CH, J_{α-CH}-_{βCH}=J_{α′-CH}-_{βCH}=13.5 Hz); 7.10 (d, 1H, ArH IA, J=8.5 Hz); 7.40 (dd, 2H, H7 and H7′, J_{H7-H4}=4.5 Hz, J_{H7-H6}=8.5 Hz); 7.68 (m, 2H, H6 and H6′); 7.76 (dd, 1H, ArH IA, J=2 Hz, J=8.5 Hz); 7.81 (dd, 2H, H4 and H4′, J_{H4-H6}=1 Hz, J_{H4-H7}=5 Hz); 8.29 (d, 1H, ArH IA, J=2 Hz); 8.35 (t, 1H, vinyl β-CH, J_{β-CH}-_{α-CH}=J_{β-CH}-_{α′-CH}=13.5 Hz), 11.64 (s, 1H, ArSO₃H).

### EXAMPLE 1.8

### Synthesis of 1-methyl 5-Cy3-isatoic anhydride (13)

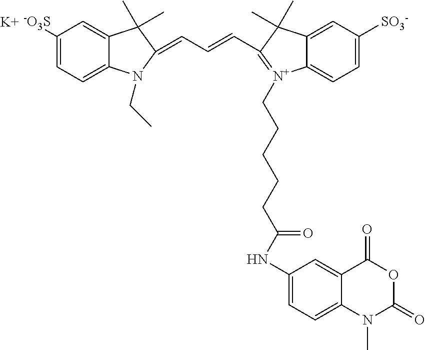

In a 25 mL round-bottomed flask, 500 mg of the conjugate Cy3-IA 7 (0.63 mmoles; 1 eq) are placed in solution in 2.6 mL of DMF. 175 mg of K₂CO₃ (1.26 mmoles; 2 eq) as well as 198 μL of iodomethane (3.16 mmoles; 5 eq) are then added and the reaction is left under stirring at ambient temperature. LC-MS monitoring shows that the starting product is completely consumed after 1 h. After 1 h of reaction, the reaction medium is evaporated until dry then purified on a C18 silica gel column by using an eluent gradient (2-propanol/DMF 95:5 then 2-propanol/DMF 50:50), then dried under vacuum in order to eliminate the residual DMF. (Since the product is not soluble in 2-propanol/DMF 95:5 mixture, a solid deposit is prepared in the DMF). The final product is obtained in the form of a pink powder with a yield of 95% (505 mg; 0.6 mmoles). An ionic chromatography analysis showed that the product is obtained in the form of a monopotassium salt.

M+H=843 g·mol⁻¹, TLC: (H₂O/DMF 8/2; UV development) Rf=0.1

LC/MS: condition 1: RT=7.8 mins; max absorption λ=267.6; 518.4 and 551.2 nm; [M+H]⁺: 805.6.

¹H NMR (500 MHz, DMSO-d₆): δ 1.31 (t, 3H, β′-CH₃, J_{β′-CH3-α′-CH2}=7 Hz); 1.44 (m, 2H, γ-CH₂); 1.68-1.71 (m, 14H, δ-CH₂+2×C(CH₃)₂); 1.79 (m, 2H, β-CH₂); 2.34 (t, 2H, ε-CH₂, J_{ε-CH2-δ-CH2}=7 Hz); 3.45 (s, 3H, NCH₃); 4.16 (m, 2H, α-CH₂ and α′-CH₂); 6.66 (2d, 2H, vinyl α-CH and vinyl α′-CH, J_{α-CH}-_{β-CH}=J_{a′-CH}-_{β-CH}=13.5 Hz); 7.42 (m, 3H, ArH IA+H7 and H7′); 7.68 (m, 2H, H6 and H6′); 7.76 (m, 3H, ArH IA+H4 and H4′); 8.36 (m, 2H, ArH IA+vinyl β-CH).

### EXAMPLE 1.9

### Synthesis of 3-(2-(2-(3-biotin-dPEG₃-propanamido)ethyl)disulfanyl)propanoic acid (22)

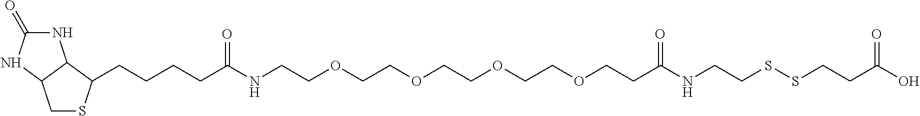 [SMILES: O=C(O)CCSSCCNC(=O)CCOCCOCCOCCOCCNC(=O)CCCCC1SCC2NC(=O)NC21]

NHS—S—S-dPEG₄-biotin (1.390 g; 1.849 mmol; Quanta Biodesign, Powell, USA ref.: 10194) is placed in solution in 17.56 mL of DMF (52 mM), and introduced into a 100 mL round bottomed flask. 17.56 mL of water are added and the medium is placed at 75° C. under refrigerant.

After 4 hours at this temperature, LC-MS monitoring indicates that the NHS—S—S-dPEG₄-biotin was completely hydrolysed.

After evaporation until dry, a viscous yellowish oil is obtained which is co-evaporated three times with an ACN/DMF mixture: 5 mL/3 mL in order to eliminate the remaining water, and which is then triturated three times with ether in order to dry it and eliminate the residual DMF.

The product is taken up in 2 mL of a dichloromethane/methanol/acetic acid mixture: 79/20/1, and deposited onto a normal-phase chromatographic column (silica gel 60, Fluka, ref.: 60737); the elution is carried out with the same mixture. The fractions containing 3-(2-(2-(3-biotin-dPEG₃-propanamido)ethyl)disulfanyl) propanoic acid are gathered and co-evaporated with a toluene/DMF mixture: 10 mL/5 mL in order to free them from the remaining acetic acid then evaporated until dry.

Mass obtained: 1.02 g, i.e. a yield of 84%.

M+H=655.2 g·mol⁻¹

### EXAMPLE 1.10

### Synthesis of 5-(3-(2-(2-(biotin-dPEG₃-propanamido)ethyl)disulfanyl))propanamido)isatoic anhydride (23)

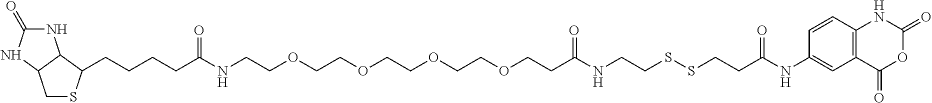 [SMILES: O=C(CCCCC1SCC2NC(=O)NC21)NCCOCCOCCOCCOCCC(=O)NCCSSCCC(=O)Nc1ccc2[nH]c(=O)oc(=O)c2c1]

3-(2-(2-(biotin-dPEG₃-propanamido)ethyl)disulfanyl)propanoic acid 22 (486 mg; 0.741 mmol; 1.0 eq) is taken up in 7.42 mL of DMF (100 mM solution) and introduced into a 25 mL two-neck round-bottomed flask placed under argon. The round-bottomed flask is cooled to 0° C. in an ice bath, then N-methylmorpholine (106 μL; 0.965 mmol; 1.3 eq) and isobutyl chloroformate (126 μL; 0.965 mmol; 1.3 eq) are added to the medium. After 15 minutes under stirring at 0° C., 5-amino isatoic anhydride 1 (172 mg; 0.965 mmol; 1.3 eq) in a 200 mM DMF solution (4.82 mL) is introduced at ambient temperature.

After 1 hour at ambient temperature, the round-bottomed flask is put back an ice bath and a second addition of N-methylmorpholine (106 μL; 0.965 mmol; 1.3 eq) and isobutyl chloroformate (126 μL; 0.965 mmol; 1.3 eq) is carried out. After 15 minutes at 0° C., the ice bath is removed and 2.41 mL of the 200 mM 5-amino isatoic anhydride stock solution in DMF (86 mg; 0.482 mmol; 0.65 eq) are added.

After an extra 20 minutes under stirring at ambient temperature, the round-bottomed flask is put back an ice bath, and a last addition of N-methylmorpholine (106 μL; 0.965 mmol; 1.3 eq) and isobutyl chloroformate (126 μL; 0.965 mmol; 1.3 eq) is carried out. After 15 minutes at 0° C., the ice bath is removed and the reaction is left under stirring for an extra 30 minutes.

The medium is evaporated until dry after addition of one spatula of reverse-phase silica (RP18 silica/40-63 μm, Merck LiChroprep), then co-evaporated three times with an acetonitrile/DMF mixture: 50/50, until a very dry chestnut-brown powder is obtained.

The solid is then deposited onto a reverse-phase chromatographic column with a diameter of 2.5 cm and a height of 15 cm (RP18 silica/40-63 μm, Merck LiChroprep) and eluted with the following successive mixtures: DMF/water: 50/50, then DMF/water: 60/40, then DMF/water: 75/25, and finally DMF/water: 80/20.

Finally, the medium is evaporated until dry and co-evaporated three times with 7 mL of acetonitrile. The product appears in the form of orange crystals.

Mass obtained: 220 mg, i.e. a yield of 36%.

M+H=815.3 g·mol⁻¹

NMR ¹H (200 MHz, DMSO): 1.0 to 1.75 (m, 7H, 11-11-12+1H unattributed); 2.08 (t, 2H, 13); 2.32 (t, 2H, 30); 2.75 to 2.85 (m, 4H, DMSO+38); 2.86 (s, 8H, DMF); 2.82 to 2.98 (m, 9H, 6-8-17+2H unresolved); 3.02 to 3.44 (m, 11H, unresolved cluster); 3.45 to 3.55 (m, 12H, 20-21-23-24-26-27 i.e. the PEG chain); 3.55 to 3.65 (m, 4H, 18-29); 4.22 (2m, 2H, 3-4); 6.43 (2s, 2H, 1-5); 8.0 (m, 3H, 32-15+1H unresolved).

### EXAMPLE 1.11

### Synthesis of N-methyl 5-(3-(2-(2-(biotin-dPEG₃-propanamido)ethyl)disulfanyl))propanamido) isatoic anhydride (24)

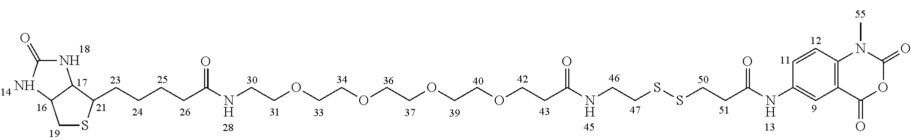 [SMILES: Cn1c(=O)oc(=O)c2cc(NC(=O)CCSSCCNC(=O)CCOCCOCCOCCOCCNC(=O)CCCCC3SCC4NC(=O)NC43)ccc21]

5-(3-(2-(2-(biotin-dPEG₃-propanamido)ethyl)disulfanyl))propanamido)isatoic anhydride 23 (50 mg; 0.061 mmol; 1.0 eq; 2.49 mL of 24.2 mM solution in DMF) is placed into a 25 mL round-bottomed flask under stirring in an ice bath. Potassium carbonate (9.3 mg; 0.067 mmol; 1.1 eq) in suspension in DMF is added immediately, and the medium is left under stirring at 0° C. for 5 minutes.

Iodomethane (15.2 μL; 0.244 mmol; 4.0 eq) is then introduced into the medium, and the reaction is left under stirring at 0° C. for 45 minutes.

In order to free the obtained product from the remaining potassium carbonate, it is deposited onto a reverse-phase chromatographic column with a diameter of 1 cm and a height of cm, (RP18 silica/40-63 μm, Merck LiChroprep) and eluted with 100% acetonitrile.

After evaporation of the medium until dry, the product is triturated four times with 5 mL of ether. The product appears in the form of yellow flakes.

Mass obtained: 55.7 mg (the excess mass is due to the presence of trapped DMF in the final product).

M+H, 829.3.mol⁻¹

NMR ¹H (200 MHz, DMSO): 1.2 to 1.75 (m, 6H, 23-24-25); 2.085 (t, 2H, 26); 2.338 (t, 2H, 43); 2.5 to 2.7 (m, 2H, 51); 2.75 to 2.95 (m, 24H, 55-19-47-50+16H unattributed—possibly due to DMF); 3.05 to 3.30 (m, 6H, 19 and 5H unattributed); 3.3 to 3.7 (m, 41H, 21-46 and 30-31-33-34-36-37-39-40-42 (PEG chain)+20H unattributed possibly due to residual water); 4.150 and 4.319 (m, 2H, 16-17); 6.418 (d, 2H, 14-18); 7.477 (d, 1H, 11); 7.887 (t, 1H, 45); 7.976 (m, 3H, 13+2H unattributed possibly due to DMF); 8.091 (t, 1H, 28); 8.363 (d, 1H, 12); 10.389 (s, 1H, 9).

NB: a total of 95 protons are detected instead of the 58 expected for this molecule. The extra protons form three main clusters, and correspond to water and DMF respectively.

### EXAMPLE 2

### Synthesis of Derivatives of Isatoic Anhydride Conjugated to a Group of Interest Borne by Intracyclic Nitrogen of Isatoic Anhydride

This example illustrates the evaluation of another isatoic anhydride functionalisation site, which appears not to be on the aromatic ring. For this purpose we chose to use the alkylation reaction of intracyclic NH of isatoic anhydride, described previously, to conjugate a molecule of interest therein. Thus this position may be alkylated by an electrophilic derivative of a compound of interest, such as a halogen derivative of biotin, such as described in FIG. 4.

Iodoacetyl-LC-Biotin 17 or the biotinylated compound 20 is reacted with isatoic anhydride 1 in the presence of K2CO3 or DIPEA to form, by alkylation of the —NH—, the biotinylated compound 18 or 21.

This method is not limited to unsubstituted isatoic anhydride, it is indeed possible to modulate the reactivity of the isatoic anhydride part to nucleophiles (such as for example the 2′OH hydroxyls) by appropriately substituting the aromatic ring. The reference from Weeks 2008 JACS pages 8884 describes that a nitro IA Me has a reactivity of around 40 times greater than that of IA Me due to the attracting effect of NO2. The person skilled in the art thus has a very precise idea of the substituting groups to be used to thus increase or lower the reactivity of isatoic anhydride.

To illustrate the effect of the substitution of the aromatic part on the reactivity of isatoic anhydride, this example describes the synthesis of N(Biot PEG2) acetamido IA (19) compound by reaction of iodoacetyl-PEG2-Biotin (17), with 5-acetamido IA (16) (synthesised by acetylation of 5-amino IA 15).

It should be noted that it would also be possible for the isatoic anhydride coupled to the molecule of interest to be synthesised by constructing an appropriately functionalised anthranilate, and then closing the isatoic anhydride with an equivalent of phosgene.

### EXAMPLE 2.1

### Synthesis of 1-biotin-ethylene glycol-isatoic anhydride (18)

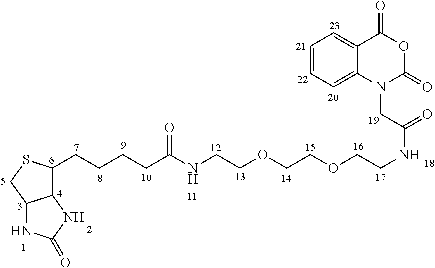 [SMILES: O=C(CCCCC1SCC2NC(=O)NC21)NCCOCCOCCNC(=O)Cn1c(=O)oc(=O)c2ccccc21]

Isatoic anhydride 15 (45.1 mg; 276.6 μmol; 3.0 eq) is dissolved in DMF (460 μl; 600 mM). Potassium carbonate (38.2 mg; 276.6 μmol; 3.0 eq) is suspended in this mixture. After 5 minutes of stirring in a vortex at 1000 rpm, the device is placed in a cold chamber at 4° C. before feeding iodoacetyl-PEG2-biotin 17 (50 mg; 92.2 μmol; 1.0 eq, Pierce-Thermoscientific, Whaltham, USA) in solution in DMF (730 μl; 126 mM; 1.3 eq). After 90 minutes, the iodoacetyl-PEG2-biotin has disappeared and the reaction has finished. The tube is centrifuged and the supernatant recovered. The pellet is washed twice with 200 μL of DMF. The supernatants are assembled and one spatula of RP18 silica is added before the solvent is evaporated until dry. The powder obtained is purified by reverse-phase chromatography on a column with a diameter of 1 cm and a height of 10 cm. The eluent is a water/ACN/DMF mixture: 79/20/1.

Mass obtained=11.8 mg, i.e. a yield of 22% (purity of around 60%)

M+H, 578.2 g·mol⁻¹

### EXAMPLE 2.2

### Synthesis of 5-acetamido-isatoic anhydride (16)

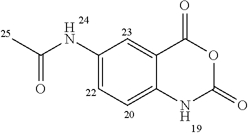 [SMILES: CC(=O)Nc1ccc2[nH]c(=O)oc(=O)c2c1]

5-amino-isatoic anhydride (740 mg; 4.15 mmol; 1.0 eq) is dissolved in DMF (21 ml; 200 mM) and stirred for two minutes before acetic anhydride (395 μl; 4.15 mmol; 1.0 eq) is added. After 3 hours and 40 minutes, the reaction is finished. The product formed is precipitated in 210 ml of water then spin-dried on sintered glass of porosity 3. The residue obtained is then dried in the oven. Mass obtained=753 mg, i.e. a yield of 82.4%.

M+H, 221.0 g·mol⁻¹

### EXAMPLE 2.3

### Synthesis of 1-biotin-diethyleneglycol-5-acetamido-isatoic anhydride (19)

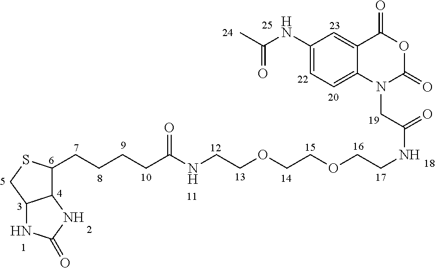 [SMILES: CC(=O)Nc1ccc2c(c1)c(=O)oc(=O)n2CC(=O)NCCOCCOCCNC(=O)CCCCC1SCC2NC(=O)NC21]

5-acetamido-isatoic anhydride (49.5 mg; 224.8 μmol; 2.5 eq) is weighed in an 8 ml flask, to which potassium carbonate (82.1 mg; 594 μmol; 6.6 eq) is added. DMF (2.65 ml; 100 mM) is added to this mixture. The medium is stirred for two minutes then placed at −25° C. In parallel, a solution of iodoacetyl-PEG2-Biotin (Pierce-Thermoscientific, Whaltham, USA) (48.6 mg; 89.6 μmol; 1.0 eq) is prepared in DMF (895 μl). The iodoacetyl-PEG2-Biotin solution is added into the reaction medium and stirred in a vortex for 16 hours at 800 rpm in a cold chamber at 4° C. The reaction medium is filtered on paper and the filtrate is purified by reverse-phase chromatography with solid deposit. The column has a diameter of 1 cm and a height of 10 cm. The eluent is a water/ACN mixture: 80/20. Mass obtained=15 mg, i.e. a yield of 10.5% (purity of less than 50%)

M+H, 635.2 g·mol⁻¹

### EXAMPLE 2.4

### Synthesis of N-(biotin-dPEG₃-propyl)-6-bromohexanamide (20)

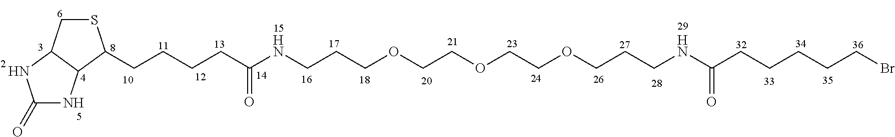 [SMILES: O=C(CCCCCBr)NCCCOCCOCCOCCCNC(=O)CCCCC1SCC2NC(=O)NC21]

In a 50 mL round-bottomed flask, 317 mg of 6-bromo caproic acid (1.624 mmol; 1.3 eq) are co-evaporated twice in succession with 5 mL of DMF. The 6-bromo caproic acid is taken up in 20 mL of DMF (81.2 mM) and transferred into a 100 mL two-neck round-bottomed flask under argon. N-methylmorpholine (316 μl; 2.873 mmol; 2.3 eq) and isobutyl chloroformate (212 μl; 1.624 mmol; 1.3 eq) are then added to the medium after cooling to 0° C. in an ice bath. After 5 minutes at 0° C. under stirring, Biotin-dPEG₃-NH₃⁺-TFA⁻ (0.7 g; 1.249 mmol; 1.0 eq, Quanta Biodesign, Powell, USA, ref.: 10193) in solution in 6.235 mL of DMF (200 mM) is added to the medium at ambient temperature. After 30 minutes under stirring, the content of the round-bottomed flask is evaporated until dry and co-evaporated twice with 10 mL of acetonitrile. 1.332 g of a yellowish and very viscous oil, taken up in 30 mL of dichloromethane, is then obtained. The organic phase is washed twice with 20 mL of 10 mM HCl in order to remove the residual Biotin-dPEG₃-NH₃⁺-TFA, then twice with 20 mL of saturated NaHCO₃ solution, and finally twice with 20 mL of NaCl (brine). After evaporation of the dichloromethane, 434 mg of a solid is obtained which is taken up in 3 mL of dichloromethane and deposited onto a normal-phase chromatographic column with a diameter of 3 cm and a height of 15 cm, (silica gel 60, Fluka, ref.: 60737, Saint Louis, USA). The products are eluted with a dichloromethane/methanol mixture: 80/20 at a speed of 5 cm/min, which makes it possible to get rid of one of the three coproducts formed during the reaction. 306 mg of a yellowish very viscous oil is obtained after evaporation until dry. This is triturated with ether three times until a white powder is obtained.

Mass obtained: 120 mg, i.e. a yield of 15%.

M+H, 623.3 g·mol⁻¹

NMR ¹H analysis (200 MHz, DMSO): 1.25 to 1.9 (m, 18H, 10-11-12-32-33-34-35-17-27); 2.07 (t, 4H, 13-32); 3.0 to 3.2 (m, 5H, 8-16-28); 3.25 to 3.60 (m, 14H, 18-20-21-23-24-26-36); 4.1 to 4.4 (m, 2H, 3-4); 6.4 (2s, 2H, 2-5); 7.8 (m, 2H, 15-29).

### EXAMPLE 2.5

### Synthesis of N-(N-(biotin-dPEG3-propyl)-6-bromohexanamide) isatoic anhydride (21)

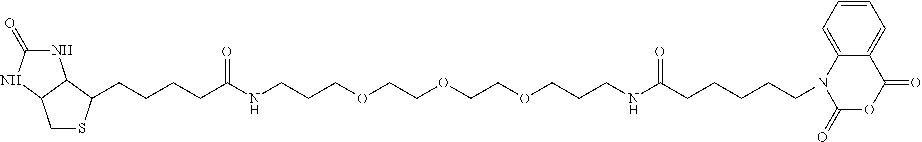 [SMILES: O=C(CCCCCn1c(=O)oc(=O)c2ccccc21)NCCCOCCOCCOCCCNC(=O)CCCCC1SCC2NC(=O)NC21]

N-(biotin-dPEG₃-propyl)-6-bromohexanamide ((20); 280 mg; 0.449 mmol; 1.0 eq) and isatoic anhydride (15); 110 mg; 0.674 mmol; 1.5 eq) are coevaporated twice with DMF and taken up in 2 mL of DMF. The two reagents are introduced into a 25 mL round-bottomed flask and the medium is evaporated until dry. The round-bottomed flask is placed under refrigerant and heated to 120° C. before adding N-ethyldiisopropylamine (768 μL; 5.467 mmol; 12.2 eq). The medium rapidly turns bright yellow. After 45 minutes at 120° C., the pH of medium is taken from 9 to a value of between 5 and 6 by successive additions of 0.015N HCl, in order to prevent the opening of the isatoic anhydride, favoured in a basic medium. The product is then deposited onto a reverse-phase column with a diameter of 1.5 cm and a height of 15 cm (RP18 silica/40-63 μm, Merck LiChroprep). The elution is carried out with the following mixtures: ACN/water: 10/90, then ACN/water:25/75, and finally ACN/water:50/50.

Mass obtained: 37 mg, i.e. a yield of 11.6%.

M+H, 706.3 g·mol

### EXAMPLE 3

### Demonstration of the Reactivity of an Isatoic Anhydride Derivative Conjugated to Cyanine 3 (Cy3 IA Me, 13), to a Model Oligoribonucleotide (ORN) with 27 Bases. Measure of the Regiospecificity of the Acylation and of the Functionalisation Rate

We evaluate the reactivity of isatoic anhydride derivatives to RNA, and therefore demonstrate the selective functionalisation of a model ORN with 27 bases (seq: 5′-AAC-CGC-AGU-GAC-ACC-CUC-AUC-AUU-ACA-3′ Eurogentec, Liege, Belgium). For this purpose, a fixed quantity of ORN is reacted with an isatoic anhydride derivative conjugated to a molecule of interest (Cy3 IA Me (13)), under various temperature conditions, duration conditions, reaction medium conditions, etc. HPLC monitoring of the reaction at 260 nm makes it possible to detect the disappearance of the peak corresponding to the initial ORN and the appearance of peaks corresponding to acylated ORN. These products have a greater retention time and have an absorption spectrum corresponding to both that of ORN and that of the fluorescent label.

The appearance of these peaks is therefore itself the demonstration of the functionalisation of the ORN. However, for greater precision on the functionalisation site and to precisely evaluate the functionalisation rate (Fr), the ORN population (labelled and unlabelled) is separated from the excess label by precipitation with acetone/lithium perchlorate or by gel permeation (NAP 5™, GE Health Care. Then the ORNs thus obtained are submitted to hydrolysis with the nuclease P1 (Aldrich, Saint Louis, USA) and with the alkaline phosphatase (Aldrich, Saint Louis, USA), in order to hydrolyse the phosphate diester bonds of ORN.

An LC-MS analysis (chromatographic chain coupled to a mass spectrometer) of this mixture makes it possible to characterise and identify several populations as described in FIG. 5:
the unlabelled ribonucleosides
the labelled mono-nucleoside adducts which differ very distinctly by a greater retention time and by a UV spectrum characteristic of the nucleic acids and of the label (in the case of Cy3 for example)
dinucleotides acylated at 2′ which, due to the anthranilate group substituted by the group of interest at 2′ cannot be cleaved by nuclease P1. The resistance to the nucleases of the phosphate diester bond, when a voluminous group is introduced at 2′, is well known to the person skilled in the art.
peracylated 2′OH trinucleotides or quadrinucleotides which may theoretically form, but which are statistically very poorly represented (because it would then be necessary for there to be two or three consecutive acylations of the 2′OH), are not sought.

It should be noted the acylated 5′O derivative is only very poorly represented, and is not visible as described by Servillo (Eur. J. Biochem. 1993 583-589).

The detection of these different labelled nucleoside or nucleotide adducts make it possible to demonstrate the functionalisation, on the hydroxyls at 2′ and on the end of the ORN (position 3′ and 2′).

The ORN functionalisation rate (Fr) is evaluated by measuring:
the area corresponding to the terminal mononucleoside adducts (functionalised at 2′ and at 3′) and the area corresponding to the acylated dinucleosides (functionalised internally on 2′OH)
compared to the area of the peaks corresponding to the four ribonucleosides.

The internal/external regiospecificity of the acylation is evaluated by measuring the relative proportion of the areas corresponding to the:
end mono-nucleotides acylated at 2′ and at 3′
and di-nucleotides acylated internally on 2′OH
which are easily detectable by HPLC.

The measurement of the ORN functionalisation rate and of the external/internal regiospecificity of the acylation are described in FIG. 6 (Graph A: native ORN with 27 bases. Graph B: ORN functionalised with compound 13 and precipitated. Graph C: ORN functionalised with said compound 13, precipitated and enzymatically hydrolysed. Graph D: enlargement of graph C with the study of the nucleoside adducts or functionalised nucleotides (peak 1 corresponds respectively to the acylated dinucleotides (42%), peaks 2 and 3 to the nucleosides acylated at 2′ or at 3′ (58%) and peak 4 to the nucleosides acylated at 2′ and at 3′. The * represents a dinucleotide or a nucleoside functionalised with a molecule of Cy3-NMe anthranilate. The entirety of the peaks 1, 2, 3 and 4 makes it possible to measure the functionalisation rate).

Operating Mode:

In a standard experiment, the equivalent of 8 nmol of an ORN with 27 bases (5′-AAC-CGC-AGU-GAC-ACC-CUC-AUC-AUU-ACA-3′ (Eurogentec, Liege, Belgium) in solution in water are dried beforehand with a centrifugal evaporator (RCT 60, Jouan, St Herblain, France) in a 2 mL plastic Ependorf tube. 40 μL of water is then added to the dry residue, in order to solubilise the ORN, then 20 μL of a buffered solution (in the example a Triethylammonium acetate buffer solution, pH 7, 1M Aldrich, St Louis, USA ref.: 09748-100 ml) and finally 20 μL of a 120 mM solution of an isatoic anhydride derivative functionalised with a group of interest in DMSO (i.e. the molecule Cy3 IA Me (13)) in this example). The mixture is incubated for 60 minutes at 65° C. in an oven on a rack which is temperature-balanced in advance. An HPLC injection (conditions 3) makes it possible to monitor a formation of a certain quantity of acylated ORN as described in FIG. 6. To eliminate the salts and the excess label, the mixture is then purified on Sephadex NAP 5 gel (GE Health Care, Uppsala, Sweden)) and/or triple precipitation in 1.2 mL of a 180 mM Acetone/LiClO4 75/25 mixture. To finish, the pellet is dried with acetone and is evaporated with the centrifugal evaporator (RCT 60, Jouan, St Herblain, France). The residue is then taken up in 33 μL of a H₂O/DMSO 85/15 solution and an HPLC injection (conditions 3) is performed in order to verify the proper elimination of the label. There is added to the mixture 2 μL of nuclease P1 (ref.: N8630 Sigma-Aldrich, St Louis, USA) in 1 U/μl solution in its buffer: 20 mM sodium acetate pH 5.5; 1 mM ZnCl₂; 50 mM NaCl and 1 μL of 7 u/μl alkaline phosphatase in water (Ref. P7923-2KU Sigma-Aldrich, St Louis, USA). The medium is left at ambient temperature for 4 to 6 hours. It is then injected into HPLC (conditions 3) in order to verify the total hydrolysis of the ORN and in order to analyse the nature of the fragments obtained in accordance with what was described above and as described in FIG. 6.

1—Results: The LC-MS analysis of a chromatogram characteristic of this example makes it possible to identify the different types of products listed below (HPLC conditions 3): The unacylated ribonucleosides Cytosine (C), Uracil (U), Guanine (G) and Adenine (A) (Rt of 1.5-5 mins) which are easily identifiable by mass spectrometry and by their UV profile. Their presence is explained by the fact that the ORN is only subjected to a controlled acylation which does not acylate all of the nucleosides. Nuclease P1 may then cleave the bonds between unacylated nucleosides.

2—The dinucleotides acylated at 2′ which, being insensitive to the action of the nuclease P1, possess a greater retention time (Rt of 11.8-12.5 min). They can be identified by mass spectrometry and by UV thanks to the absorption band characteristic of anthranilate, of nucleoside and of Cy3 (respectively 260, 360 and 550 nm). Since the ORN sequence which acts as the model is the following: 5′-AAC-CGC-AGU-GAC-ACC-CUC-AUC-AUU-ACA-3′, it is important to understand that after controlled labelling and hydrolysis of the ORN, only a certain number of dimers, which are dependent on the sequence, may be obtained. Thus, by shifting along the ORN sequence, it is foreseeable to detect the 2′-acylated dimers: 5′-AA, AC, CC, CG, GC, CA, AG, GU, UG, GA, AC, CA, AC, CC, CC, CU, UC, CA, AU, UC, CA, AU, UU, UA, AC and CA-3′. Since the GG base pair cannot be found in the sequence, it will not actually be found in LC-MS.

3—The acylated riboadenine at 2′, at 3′ or in both positions 2′ and 3′, which possesses an even greater retention time (Rt of 12.7-13.8 mins), because it does not possess a phosphate diester link. These adducts can be identified by mass spectrometry and by UV thanks to the absorption band characteristic of the nucleoside, anthranilate and Cy3 (respectively 260, 360 and 550 nm). The fact that only one single type of labelled nucleoside (only rA) is detected indicates that the phosphate diester link is perfectly stable if the 2′-OH group is acylated with an isatoic anhydride derivative. Indeed, if it was sensitive to hydrolysis by nuclease P1, the four labelled nucleoside adducts would be formed due to random labelling on the entirety of the oligoribonucleotide chain. The presence of the acylated rA adduct is therefore explained by the labelling of the nucleoside situated at the 3′ end of the ORN (rA), which is detached from the rest of the chain during hydrolysis with NP1.

A specific study, by mass spectrometry, of the mass of all of these fragments together with the mass of the anthranilate linked to the Cy3 indeed makes it possible to demonstrate their formation as indicated in FIGS. 6 and 7.

The functionalisation rate is evaluated with UV at 260 nm by integration of the clusters corresponding to the acylated dinucleotides and to the acylated nucleosides) relative to the clusters corresponding to the four ribonucleosides. The ratio of the population of acylated dinucleotides to the population of acylated nucleosides is evaluated in the same way in order to evaluate the percentage of functionalisation on the 3′ terminal end.

Comment:

The functionalisation rate values, presented within the framework of this invention, take into account the correction factor which must be applied according to the molar extinction coefficient at 260 nm of each of the nucleosides and according to whether they are nucleosides or dinucleotides. The areas measured are thus corrected according to:
the molar epsilons at 260 nm of rC, rU, rG and rA for the unacylated nucleosides,
an average value of the molar epsilons at 260 nm of 5′AA, AC, CC, CG, GC, CA, AG, GU, UG, GA, AC, CA, AC, CC, CC, CU, UC, CA, AU, UC, CA, AU, UU, UA, AC and CA 3′ for the acylated dinucleotides, and
the molar epsilons at 260 nm of rA for the acylated mononucleosides (see Table 2).

TABLE 2
Molar epsilon values of the different acylated mononucleosides
and dinucleotides formed during enzymatic hydrolysis
of an ORN functionalised by Cy3 IA Me (13)
Average epsilon of the mononucleosides and dinucleotides

| | | Epsilon weighted according |
|---|---|---|
| | Molar epsilon | to the sequence of the ORN |
| | (mol⁻¹ · l · cm⁻¹) | (mol⁻¹ · l · cm⁻¹) |
| rA | 13700 | |
| rC | 7300 | |
| rG | 10800 | |
| rU | 8400 | |
| AA | 27400 | 27400 |
| AC*9 | 21000 | 189000 |
| CG*3 | 18100 | 54300 |
| UG*2 | 19200 | 38400 |
| CC*3 | 14600 | 43800 |
| AG*2 | 24500 | 49000 |
| UU | 16800 | 16800 |
| AU*3 | 22100 | 66300 |
| UC*3 | 15700 | 47100 |
| Total dimers = 27 | | |

| | |
|---|---|
| Average weighted dinucleotide Epsilon | 19707 |

Discussion and Conclusion:

The functionalisation rate corrected to 4.5% and the internal/external regiospecificity of acylation is evaluated at 58/42 (see entry 7 in Table 3 in example 5 hereafter). This means that in these conditions around five nucleosides will be labelled on average every 100 bases, i.e. one label every 20 bases. This is perfectly consistent with the labelling rates recommended to respect a compromise between a label number sufficient for good detection and a hybridisation which is not impacted by an excessively large number of bulky groups.

Similarly, a ratio of 58/42 indicates that the 3′ end is thirty-six times more reactive than the positions at 2′ (0.58×1)/(0.42/26). This is consistent with the data from the literature which demonstrates a greater reactivity of the 3′ end (Nawrot Nucleosides and Nucleotides 1998 815-829).

With this example, we demonstrate the functionalisation of an ORN by a fluorescent derivative of isatoic anhydride.

### EXAMPLE 4

### Demonstration, Via a MALDI T of Mass Spectrometry Technique, of the Functionalisation of an ORN with 27 Bases by Cy3 IA Me (13).

Objective:

We demonstrate that an ORN reacted with an isatoic anhydride derivative, Cy3 IA Me (13), does lead to an acylated ORN bearing several Cy3-anthranilate adducts.

Operating Mode:

Exactly the same protocol is followed as in example 3, but the ORN is functionalised either with 15 mM or with 30 mM of Cy3 IA ME (13) in order to give the corrected functionalisation rates of 2.6 and 4.2%, respectively. A portion of the functionalised and non-hydrolysed ORNs is mixed with a mixture of 3-hydroxy picolinic acid and diammonium citrate in proportions 9/1, then analysed by MALDI TOF mass spectrometry (Bruker, Billerica, Mass., USA). Analysis of the spectrograms then makes it possible to detect M+H adducts at 8519.21; 9281.14 and 10043.07 dalton, which respectively correspond to the ORN and to the ORN functionalised once and twice with an anthranilate-Cy3 adduct (FIG. 8). The numbers are expressed in dalton. Graph 1 corresponds to the analysis of the ORN alone and graphs 2 and 3 correspond to the same ORN, functionalised with 15 and 30 mM of compound 13, respectively.

### Results and Conclusion:

This provides direct evidence that isatoic anhydride derivatives react on the ORN and that the number of adducts depends on the concentration of functionalisation reagent. Since the chemical nature of an ORN is strictly the same as an RNA strand, we can be certain that an RNA strand will be functionalised in the same manner as is further demonstrated in examples 9 and 12 of this invention application.

### EXAMPLE 5

### Demonstration of the Reactivity of Other Isatoic Anhydride Derivatives Conjugated to Molecules of Interest, to a Model Oligoribonucleotide (ORN) with 27 Bases. Measurement of the Functionalisation Rate

Objective:

Demonstrate the reactivity of other isatoic anhydride derivatives conjugated to a molecule of interest, i.e.:
5Biot IA 5,
5Biot IA Me 9,
5Biot IA SO₃⁻¹⁰,
5Biot PEG4 IA 6,
5Biot PEG4 IA Me 11,
Cy3 IA 7,
Cy3 IA Me 13,
N(Biot PEG2) IA 18,
N(Biot PEG2) acetamido IA 19,
N(Biot PEG4) IA 21
5Biot PEG4 SS IA 23, and
5Biot PEG4 SS IA Me 24, to a model ORN with 27 bases, following the labelling protocol described in example 3.

Operating Mode:

Briefly; an ORN with 27 bases (8 nmoles) is incubated for 1 hour at 65° C. in 30 mM solution with any one of compounds 5, 9, 10, 6, 11, 7, 13, 18, 19, 21, 23, or 24 in the presence of 250 mM TEAAc pH 7 and DMSO at 25% in the mixture. The labelled ORN is then purified and treated as in example 3. From this, Table 3 below is produced, which indicates the functionalisation rate and the external/internal regiospecificity of acylation, according to the different isatoic anhydride derivatives. The solubility of some of these compounds has also been determined by following the same protocol as in the preceding patent application filed by the Applicant and published under number: WO-A-2010/012949.

TABLE 3
Corrected functionalisation rate and external/internal regiospecificity
of acylation of an ORN with 27 bases having reacted with
different isatoic anhydride derivatives.

| | | | | | External/ | |
|---|---|---|---|---|---|---|
| | | HPLC | | | internal | |
| | | Purity (%) | Solubility | | regio- | |
| | | (Chart | in water | Fr | specificity | Nb |
| Entry | Compounds | Max) | (mM) | (*) | (**) | exp |
| 1 | 5Biot IA 5 | 52 | 1 | 1.2 | — | 1 |
| 2 | 5Biot IA Me | 95 | 0 | 2.6 | 55/45 | 4 |
| | 9 | | | | | |
| 3 | 5Biot IA | 60 | nd | 1.6 | 60/40 | 2 |
| | SO₃⁻ 10 | | | | | |
| 4 | 5Biot PEG4 | 85 | 19 | 3.3 | 62/38 | 2 |
| | IA 6 | | | | | |
| 5 | 5Biot PEG4 | 85 | 50 | 4 | 60/40 | 1 |
| | IA Me 11 | | | | | |
| 6 | Cy3 IA 7 | 97 | >>100 | 2.6 | 61/39 | 1 |
| 7 | Cy3 IA Me 13 | 96 | >>100 | 4.5 | 58/42 | 2 |
| 8 | N(Biot | 43 | nd | 3.4 | 56/44 | 1 |
| | PEG2)IA 18 | | | | | |
| 9 | N(Biot PEG2) | 42 | nd | 1.2 | 50/50 | 1 |
| | acetamido IA | | | | | |
| | 19 | | | | | |
| 10 | N(Biot PEG4) | 25 | nd | 0.7 | 49/51 | 2 |
| | IA 21 | | | | | |
| 11 | 5Biot PEG4 | 94 | nd | 3.1 | 52/48 | 3 |
| | SS IA 23 | | | | | |
| 12 | 5Biot PEG4 | 85 | nd | 2.8 | 62/38 | 3 |
| | SS IA Me 24 | | | | | |

(*) = corrected Fr (%) as described in example 3
(**) = Uncorrected ratio between the area corresponding to acylated mononucleosides and that corresponding to acylated dinucleotides (%) as
described in example 3

Results and Conclusion:

As in the case of the compound Cy31AMe 13 of example 3, it is demonstrated that other isatoic anhydride derivatives variously conjugated to molecules of interest react with an ORN to give, after hydrolysis, acylated nucleosides or dinucleotides. The functionalisation of the ORN with an Fr of between 0.7 and 4.5% is thus demonstrated, which as we should recall is quite sufficient for labelling applications for example, since it is sought to have between one and several labels every 100 bases.

It is generally demonstrated that the methylation of the intracyclic nitrogen of the isatoic anhydride doubles the functionalisation yield (entries 1 and 2) in the case of biotin and (entries 6 and 7) in the case of Cy3. In other cases, the yield is not doubled, but at least improved (entries 4 and 5) in the case of biotin PEG4. Curiously, this is not the case for the derivatives 5 Biot PEG4 SS IA and 5 Biot PEG4 SS IA Me. We explain this by the nature of the group of interest borne by the IA, which appears to affect its reactivity.

It is also demonstrated that generally, and whatever the isatoic anhydride derivatives, the ratio of labelled mononucleosides/labelled dinucleotides remains close to 60/40 (entries 1 to 12). This proves that the 3′ terminal end is a lot more reactive than the internal 2′-OH groups, and that the molecules of interest conjugated to the isatoic anhydride do not modify the regiospecificity. The greatest reactivity of the 3′ terminal end is attributed to the presence of cis-diol 2′, 3′. It should be noted that this ratio is slightly over-estimated in favour of internal labelling because we do not apply the epsilons correction in this calculation. By way of information and generally, an uncorrected regioselectivity of external/internal acylation of 60/40 becomes equal to 70/30 after Epsilon correction.

Conversely, the conjugation of a molecule of interest on the intracyclic —NH— of isatoic anhydride, as described in example 2, seems to exhibit an increase of the internal labelling (entries 8, 9 and 10). It would therefore appear that the site of conjugation of the molecule of interest to the isatoic anhydride has an impact on the regiospecificity of acylation, as well as its size and charge (based on results which are not communicated in this patent application).

### EXAMPLE 6

### Demonstration of the RNA Vs DNA Chemoselectivity of Isatoic Anhydride Derivatives Conjugated to a Group of Interest (5-biot IA Me 9, Cy3 IA Me 13 or 5-Biot PEG4 SS IA Me)

Objective:

In order to demonstrate the selectivity of functionalisation of RNA versus DNA, a comparative test was carried out between an ODN with 53 bases and an ORN with 27 bases subjected to the action of one of the isatoic anhydride derivatives 5-biot IA Me 9, Cy3 IA Me 13 or 5-Biot PEG4 SS IA Me 24.

Operating Mode:

8 nmoles of ODN with 53 bases: Seq: 5′-AAT-TCT-AAT-ACG-ACT-CAC-TAT-AGG-GTG-CTA-TGT-CAC-TTC-CCC-TTG-TTC-TCT-CA-3′ (Eurogentec, Liege, Belgium) or 8 nmoles of an ORN with 27 bases: Seq: 5′-AAC-CGC-AGU-GAC-ACC-CUC-AUC-AUU-ACA-3′, Eurogentec, Liege, Belgium) are reacted with compounds 9 or 13 or 24 at 30 mM in a 25/75 mixture of DMSO/TEAAc buffer (250 mM pH 7) for 1 h at 65° C. After precipitation with acetone and hydrolysis with nuclease P1 and alkaline phosphatase, the hydrolysed fragments are analysed by LC-MS, as described in example 3. An example of the chromatograms monitoring the reaction of ODN or ORN with compound 13 is shown in FIG. 9, in which graphs A, B and C respectively represent ORN, ORN functionalised with compound 13 and precipitated, and ORN functionalised with 13, precipitated and hydrolysed. Graphs D, E and F represent the same thing, but with ODN.

Results and Conclusion:

The corrected functionalisation rate of ORN is evaluated at 4.5% with label 13, at 2.6% with label 9, and at 2.8% with label 24, whereas that of the ODN is evaluated at 0.1% in the three cases. In these experimental conditions, the ODN is barely functionalised, which confirms the selectivity of functionalisation of the isatoic anhydride derivatives for RNA and the absence of reactivity on the bases. Indeed, if the bases reacted on the isatoic anhydride derivatives, there would be the formation of anthranilate nucleoside adducts after reaction with ODN. It should be noted that the 0.1% of functionalisation of ODN comes from a very low reactivity of the 5′-OH and 3′-OH ends of DNA (see in this regard Nawrot Nucleosides and Nucleotides 1998 815-829).

This example demonstrates the chemospecificity of isatoic anhydride derivatives conjugated to molecules of interest for an ORN, relative to an ODN. The presence of 2′-OH groups on ORN, whereas there are none on ODN, is the source of the chemospecific reaction on ORN.

### EXAMPLE 7

### Demonstration of the Detection, on an Affymetrix DNA Chip, of Oligoribonucleotides Functionalised by 5Biot IA Me (9) or by 5Biot PEG4 IA Me (11)

Objective:

We demonstrate that oligonucleotides biotinylated by reaction with SBiot IA Me (9) or by SBiot PEG4 IA Me (11) become detectable on a DNA chip (GeneChip Human Genome U133 Plus 2.0 Array, ref. 900470, Affymetrix, St Clara, USA).

Operating Mode:

Four synthetic oligoribonucleotides with 30 bases (Eurogentec, Seraing, Belgium), respectively corresponding to a part of the sequences of the housekeeping genes DAPS P2, GAPDH P15, HSA P12 and LYS3 P7 detected by a U133 Plus 2.0 DNA chip (Affymetrix, St Clara, USA), are each placed in solution in water to 6 μM (24 μM in total).

42 μl of the solution of the four ORNs are taken, which are then mixed with 42 μl of a potassium phosphate solution (pH 7, 1M) 42 μl of ultrapure water and 42 μl of the compounds SBiot IA Me (9) or SBiot PEG4 IA Me (11) in 120 mM solution in DMSO. These are left to incubate for 60 mins in an oven at 65° C., and then for 5 minutes in ice.

To eliminate the excess reagent, 0.3 vol of 3M sodium acetate, then 0.7 volume of pure isopropanol is added. Vortex stirring is performed and immediate centrifuging is performed for 30 mins at 14,000 rpm at +4° C. The supernatant is removed and the pellet is rinsed with 50 μL of 70% ethanol. Centrifuging is performed once again for 15 mins at +4° C. (14,000 rpm). The supernatant is removed and the pellet is dried for 5 mins with the rotary evaporator (Jouan, St Herblain, France) without heating. The residue is taken up by 70 μL of ultrapure water.

The hybridisation and detection of the functionalised ORNs is carried out in accordance with the protocol described in the Affymetrix GeneChip Human Genome U133 Plus 2.0 Array kit (ref. 900470) and in the manual: GeneChip® Expression Analysis. Technical Manual With Specific Protocols for Using the GeneChip® Hybridization, Wash, and Stain Kit P/N 702232 Rev. 3.

The ORNs functionalised and mixed with hybridisation buffer are deposited onto the Genome U133 Plus 2.0 Array chip at a concentration varying between 7 nM and 70 μM in a volume of 200 μl. The hybridisation is carried out for 16 h at 45° C. on the FS 450 fluidics station (Affymetrix, Santa Clara, USA). After rinsing, the detection of the biotinylated ORNs is performed by complexing with streptavidin coupled to phycoerythrine, followed by measuring the fluorescence detected with the aid of the GeneChip® Scanner 3000 and the GeneChip® Operating Software (Affymetrix, Santa Clara, USA). The fluorescence intensities (RFU) collected after functionalisation with SBiot IA Me (9) are shown in FIG. 10, and those with SBiot PEG4 IA Me (11) are shown in FIG. 11.

Results and Conclusion:

It is noted that the fluorescence intensities induced by the 2 molecules are very similar and very substantially greater than the background noise generated by non-functionalised ORNs (in the order of 30 RFU, not shown in FIGS. 10 and 11). The difference between the four genes originates from the differences in affinity of the ORNs for their target. These experiments demonstrate that it is possible to transfer a biotin group onto an ORN by reaction between the latter and a biotinylated isatoic anhydride molecule. The presence of biotin on the ORN then makes it possible to detect if there has been a specific hybridisation with the corresponding complementary target which is immobilised on a solid support.

The usefulness and effectiveness of isatoic anhydride derivatives for the labelling of ribonucleic acids, in DNA chips technology, is thus demonstrated.

### EXAMPLE 8

### Demonstration of the Detection, on Affymetrix DNA Chip, of RNA Transcripts Biotinylated by 5Biot IA ME (9)

Objective:

We wish to perform here a demonstration similar to that of example 7 but this time using a biological sample corresponding to the reality of a test on a DNA chip. A panel of in vitro RNA transcripts will therefore be used. They are RNAs complementary to the mRNAs present in the total RNAs coming from a biological sample.

The in vitro transcripts obtained in this manner will then be biotinylated by reaction with 5Biot IA Me (9), and will be detected on a DNA chip (GeneChip Human Genome U133 Plus 2.0 Array, ref 900470, Affymetrix, St Clara, USA).

Operating Mode:

The in vitro transcripts are generated by in vitro transcription of the total RNA extracted from blood samples with the aid of the MessageAmp II kit (Reference AM 1751, AMBION, Austin, Tex.). Briefly, after extraction of the total RNA from the blood cells, a first reverse transcription step by the enzyme T7 Polymerase is performed in order to generate the DNA strand complementary to the mRNA (cDNA). The RNA strand is then digested by the RNase H before forming the double strand of cDNA by means of a DNA polymerase. Finally, the antisense RNA (in vitro transcripts) is obtained from the cDNA using the transcriptase T7.

17.6 μL of previously obtained solution of in vitro RNA transcripts (568 ng/μl), 9.4 μL of ultrapure water and 3 μL of 100 mM Zn(OAc)₂ pH 6.5 is mixed. This solution is then incubated for 15 mins at 70° C., and then placed immediately into ice.

30 μL of this solution is sampled, to which there is added 30 μL of a potassium phosphate solution (pH 7, 1M), 30 μL of ultrapure water and 30 μl of the compound SBiot IA Me (9) in a 120 mM solution in DMSO. It is incubated for 60 mins in an oven at 65° C., then placed into ice for 5 mins.

The excess reagent is then removed in the same way as in example 7 by precipitation with isopropanol. As well as depositing on an Affymetrix chip GeneChip Human Genome U133 Plus 2.0 Array, ref 900470, Affymetrix, St Clara, USA), the detection and measurement of the fluorescence. The results obtained are shown in FIG. 12.

Results and Conclusion:

Compared to the preceding example 7, a lot more genes are detected on this chip because the corresponding biotinylated targets are present in full this time (labelled IVTs). It is noted that the fluorescence intensities observed are very substantially greater than the background noise generated by the unfunctionalised target IVTs (in the order of 30 RFU, not shown in FIG. 10). The intensity difference between the different genes originates from the respective frequency of these genes in the sample. This experiment demonstrates, from a real biological sample, that it is possible to transfer a biotin group onto a panel of RNA strands by reaction between these latter and a biotinylated isatoic anhydride molecule (9). The presence of biotin on the RNA strands then makes it possible to detect whether there has been a specific hybridisation with the complementary target.

The usefulness and effectiveness of biotinylated isatoic anhydride derivatives for the labelling of natural RNA strands is thus demonstrated.

### EXAMPLE 9

### Demonstration of the Detection, on Agilent DNA Chip, of RNA Transcripts Made Fluorescent by Reaction with Cy3 IA Me (13)

Objective:

Here, we are performing a demonstration similar to that in example 8, but this time using the Cy3 IA Me (13) molecule for the functionalisation step in order to transfer a fluorescent Cy3 molecule onto the RNA. The fluorescent transcripts thus obtained are hybridised and detected directly on an Agilent DNA chip (St Clara, USA), without use of a fluorescent streptavidin molecule.

Operating Mode:

The in vitro transcripts are generated in the same way as described in example 8. In this example, we have used four concentrations of functionalisation molecule Cy3 IA Me (13) in order to determine the most appropriate concentration for detection of the most sensitive functionalised IVTs.

6 μg of in vitro transcripts are taken up in a total volume of 20 μL of a 10 mM solution of Zn(OAc)₂ pH 6.5. The solution is incubated for 15 min at 70° C. in order to partially fragment the RNA until a majority of fragments are obtained which are of a size between 25 and 200 bases. After this incubation, the solution is immediately put into ice.

Purification with isopropanol is then performed in order to eliminate zinc acetate. To do this, 6 μL of 3M sodium acetate (i.e. 30% by volume) and 14 μL of isopropanol (i.e. 70% by volume) are added. Vortex stirring is performed and centrifuging is performed for 30 mins at 14,000 rpm (20,800 g) at 4° C. The supernatant is eliminated and the RNA pellet is rinsed with 50 μL of 70% ethanol. Centrifuging is carried out for 15 mins at 14,000 rpm (20,800 g) at 4° C., then the supernatant is eliminated and drying is carried out under vacuum with the rotary evaporator before taking the residue up in 11 μL of water.

11 μL of the transcript solution is mixed with 4 μL of a 500 mM solution of NaHCO₃ pH 8-8.5, and with 5 μL of a 120, 240, 360 or 480 mM solution of Cy3 IA Me (13) in DMSO, to obtain 30, 60, 90 or 120 mM solutions of functionalisation reagent, respectively. These solutions are incubated at 65° C. for one hour.

The excess of functionalisation reagent as well as that of the different salts are eliminated by isopropanol precipitation then by acetone precipitation. To do this, 6 μL of 3M sodium acetate (i.e. 30% by volume) and 14 μL of isopropanol (i.e. 70% by volume) are added to the functionalisation solution. Vortex stirring is performed and centrifuging is performed for 30 mins at 14,000 rpm (20,800 g) at 4° C. The supernatant is eliminated and then the pellet is rinsed with 50 μL of 70% ethanol. Centrifuging is carried out again for 15 mins at 14,000 rpm (20,800 g) at 4° C., and the supernatant is eliminated.

280 μL of water, 18 μL of 3M LiClO₄ and 900 μL of acetone are added to the pellet. Vortex stirring is performed, centrifuging is performed and the supernatant is eliminated. Next, 280 μL of water, 18 μL of 3M LiClO₄ and 900 μL of acetone are added to the pellet. Vortex stirring is performed, centrifuging is performed and the supernatant is eliminated. The pellet is rinsed with 50 μL of 70% ethanol and then centrifuging is carried out for 15 mins at 14,000 rpm (20,800 g) at 4° C. The supernatant is eliminated, and the pellet is dried under vacuum with the rotary evaporator before the pellet is taken up in 20 μL of water (i.e. [in vitro transcripts]_{theoretical}=625 ng/μL).

In order to determine the quantity of functionalised in vitro transcripts recovered after these different treatments, 1.2 μl of each end solution are assayed by UV spectrophotometry at 260 nm (Nanodrop 100, Thermo Scientific Waltham, USA). An assay is also carried out at 550 nm in order to learn the Cy3 functionalisation rate of the in vitro transcripts (% of Cy3 molecules per 100 nucleosides). This value is also known under the name DoL (Degree of Labelling) and is calculated with the following equation: DoL (in %)=100×(340×[Cy3])/(1000×[RNA]). The details of this equation are described in the protocol for the Cy3-ULS kit ref. EA-023 marketed by Kreatech (Amsterdam, The Netherlands). Table 4 below describes the DoL values following the functionalisation of IVT with different concentrations of molecule Cy3 IA Me (13).

TABLE 4
DoL values obtained after function of a panel of in vitro
transcripts with different concentrations of molecule 13

| Concentration of | Cy3 assay at | | |
|---|---|---|---|
| Cy3 IA Me (13, mM) | 550 nm in | Nanodrop IVT assay | DoL |
| in the solution | pmol/μL | at 260 nm (in ng/μL) | (in %) |
| 30 | 6.7 | 271 | 0.8 |
| 60 | 13.1 | 255 | 1.7 |
| 90 | 12.3 | 207 | 2 |
| 120 | 15.2 | 192 | 2.7 |

An analysis with a Bio Analyzer 2100 (Agilent, Santa Clara, USA) capillary electrophoresis apparatus, using an RNA Nano 6000 chip, makes it possible to give a profile of the fragmented and fluorescent RNAs, and to make sure that the majority of the fragments are between 25 and 200 bases.

Following this, a certain quantity of functionalised IVT is deposited onto an Agilent Technologies (Santa Clara, USA) DNA chip in order to detect the hybridisation of the fluorescent transcripts with the probes immobilised on the glass slides. For this, we follow the protocol described in the kits from Agilent Technologies: “SurePrint G3 Human GE 8×60K Kit ref. G4851A” or “Whole Human Genome Microarray Kit, 4×44K ref. G4112F”. Briefly; 1.65 μg of functionalised IVTs are taken up in 110 μL of hybridisation buffer and deposited onto an Agilent BMX 1 glass slide chip. This chip was custom-made, for bioMérieux, and comprises the sequences corresponding to 352 genes in the form of oligonucleotides with 60 bases. These genes correspond, with a different format, to a selection of genes found on the U133 Plus 2.0 DNA chip sold by Affymetrix and described in examples 6 and 7. In the same way, there is found here a selection of genes with are very low, low, medium and high expression in a human cell.

The hybridisation was carried out on an Agilent device (ref. G2545A) at 65° C., for 17 h with a rotation speed of 10 rpm.

After washing of the excess of functionalised unhybridised transcripts, the fluorescent spots are measured on a TECAN LS 200 scanner (Mannedorf, Switzerland) equipped with a filter suitable for the detection of Cy3.

The raw images obtained are visible in FIG. 13 and, after retreatment and matching of the fluorescence intensities measured with gene expression frequency, it is possible to plot the histogram visible in FIG. 14. This latter corresponds to the median intensity of the spots observed for the negative controls and for four groups of sequences classed according to their intensity levels on an Affymetrix chip (very low, low, medium and high) i.e. the reference method).

Results and Conclusion:

The negative control corresponds to the median intensity obtained for the unfunctionalised in vitro transcripts. It is in the order of 20 RFU, i.e. a result which is entirely acceptable corresponding to the “base”. The signal intensities of the sequences in the “Very low expression” class are not greater than those corresponding to the base. This is a coherent result because the same observations appear for the reference method on the Affymetrix chip. Nonetheless, the sequences with a low presence are well detected, and the results for the other groups of sequences are equivalent to those obtained with the Affymetrix chip from example 8.

The increase in the DoL does not improve the signal dynamics. It is therefore not useful to label the in vitro transcripts at DoLs greater than 1.6%. In conclusion, a DoL of 0.8% obtained with the label Cy3-IA Me (13) in these conditions is satisfactory to attain good results on chips.

These chips thus demonstrate the specific hybridisation of in vitro transcripts functionalised with compound Cy3-IA Me (13). They also demonstrate that it is possible to directly detect RNA strands functionalised with molecule 13 without using fluorescent streptavidin molecules.

The usefulness and effectiveness of isatoic anhydride coupled to a molecule of interest in detecting the hybridisation of RNA strands made fluorescent is demonstrated once again.

### EXAMPLE 10

### Demonstration of the Increase in the Functionalisation Rate of a Panel of Total RNAs Subjected to the Action of Alkaline Phosphatase then Reacted with the Cy3-IA Me 13

Objective:

We demonstrate that a panel of RNA strands, partially fragmented by a zinc acetate solution, sees its reactivity increase with the Cy3-IA Me 13 molecule when there has been a preliminary treatment with alkaline phosphatase.

Operating Mode:

The panel of RNA strands is MAQC RNA (Stratagene Reference: #74000). This total RNA (ribosomal and messenger) is directly extracted from ten lines of human cells of different origin: brain, breast, B Lymphocyte, uterine cervix, liver, liposarcoma, macrophage, skin, testicle, Lymphocyte T.

The functionalisation protocol is the same as that described in example 9, i.e.:
12 μg of MAQC0 RNA is incubated, or not, with a Zinc acetate solution.
The mixture is precipitated with isopropanol.
7 units of alkaline phosphatase (P7923-2KU Sigma-Aldrich) are added to the mixture, and the hydrolysis of the phosphorylated ends is allowed to proceed for 4 hours at ambient temperature.
There is then added to the mixture the molecule Cy3-IA Me 13 at 15 mM in a NaHCO₃ buffer (100 mM/DMSO 75/25 pH 8-8.5), with a total volume of 80 μl. The mixture is incubated for 1 h at 65° C.
The mixture is precipitated once again with isopropanol, then twice with acetone, before the DoL is measured as described in example 9.

The values obtained are set out in Table 5 below

TABLE 5
DoL values obtained after cleaving, treating with alkaline
phosphatase (AP) and functionalisation of the MAQC RNA

| Entry | 10 mM Zn(OAc)₂ | AP Hydrolysis | DoL (in %) |
|---|---|---|---|
| 1 | 0 mins | 0 | 1.7 |
| 2 | 0 mins | 4 h | 1.5 |
| 3 | 15 mins | 0 | 1.7 |
| 4 | 15 mins | 4 h | 2.5 |

Results and Conclusion:

With or without treatment with alkaline phosphatase, there are no differences in DoL (which is the equivalent of the functionalisation rate) when MAQC RNA is not cleaved (entries 1 and 2).

After cleaving only, the DoL does not increase further (entry 3) undoubtedly because, since the 3′ phosphate ends generated are not reactive, there can be no more functionalisation.

In contrast, the combination of cleaving of 15 mins and the hydrolysis, with alkaline phosphatase, of the generated 3′ phosphate ends increases the DoL by 106% (entry 4). The reactivity is increased accordingly without doubt because the 3′ terminal ends are in the form of diol 2′,3′.

It is therefore demonstrated that it is possible to increase the functionalisation rate by treating the cleaved RNA fragments with alkaline phosphatase. It is also indirect evidence which shows the far greater reactivity of the terminal diol 2′,3′ end of the RNA compared to the 2′ hydroxyls inside the molecule.

### EXAMPLE 11

### Demonstration of a Selective Process of Functionalising Via Compound 24, Capturing, Cleaving of the Disulfide Bond and of Eluting an ORN with 27 Bases in Mixture with an ODN with 27 Bases.

Objective:

We demonstrate that it is possible, starting from an ORN/ODN mixture reacting with SBiot PEG₄ (SS) IA Me (24), to:
selectively functionalise the ORN,
capture it using streptavidin-coated magnetic particles and potentially recover the ODN for other applications,
cleave the SS link between the immobilised ORN and the particles, and
selectively elute the ORN free from ODN.

Operating Mode:

We have a 27-mer ODN (Eurogentec ref. 21438-DNA batch #2177673, Sequence: 5′-AAC-CGC-AGT-GAC-ACC-CTC-ATC-ATT-ACA-3′) in 650 μM solution in water, and a 27-mer ORN (Eurogentec ref. 21437-RNA batch #2177672, Sequence: 5′-AAC-CGC-AGU-GAC-ACC-CUC-AUC-AUU-ACA-3′) in 952 μM solution in water. 15.4 μL of ODN and 17.5 μL of ORN are mixed and topped up with 67.1 μL of ultrapure water.

After injection into HPLC, the integration of UV peaks at 260 nm, corresponding respectively to ORN and to ODN, indicates that the mixture comprises 44% ORN and 56% ODN (Injection 1, HPLC conditions No. 4, FIG. 15).

Functionalisation Step:

80 μL of the mixture (8 nmoles) are evaporated until dry and taken up in: 10 μL of water, 5 μL of isatoic anhydride Biot PEG₄(SS)IA Me (24); 2.4 mmoles), in 480 mM DMF solution, and 5 μL of Triethyl ammonium acetate buffer in 1 M solution in water. The medium is placed in the oven at 65° C. for one hour.

Precipitation Step:

The medium is then subjected to a triple precipitation with lithium perchlorate LiClO₄ and with acetone, in order to free it from the residual BiotPEG₄ (SS) IA Me: 18 μL of LiClO₄ in 3M solution in water and 262 μL of water are added to the medium. After vortex stirring, 900 μL of acetone are added, then the medium is agitated once again and centrifuged (5 minutes at 13,000 rpm at ambient temperature). The supernatant is then removed carefully and the same method is repeated twice more with 18 μL of LiClO₄ and 282 μL of water. Finally, the medium is washed with 900 μL of acetone before stirring and centrifuging. The greater part of the supernatant is removed carefully before drying in the rotary evaporator.

The functionalisation and precipitation steps described above are repeated, starting from the dry residue taken up in 10 μL of water.

The ORN/ODN mixture is then taken up in 33 μL of water and 5 μL of the solution are then injected into HPLC (Injection 2, HPLC conditions No. 4, FIG. 15).

The following are successively introduced into a 250 μL tube:
250 μg (i.e. 50 μL of solution) of Merck MagPrep-25 streptavidin-coated magnetic particles (Merck, Darmstadt, Germany), washed twice in advance with 200 μL of PBS buffer (0.01 M PO₄⁻, 0.0027 M KCl, 0.137 M NaCl, pH=7.4 at 25° C., Sigma-Aldrich ref., Saint Louis, USA).
10 μL of PBS and 4.0 μL of precipitated ORN/ODN mixture which has reacted with the compound Biot PEG₄ (SS) IA Me (24).
The medium is left under gentle stirring at ambient temperature for 10 minutes.

After magnetisation of the particles, the supernatant is removed carefully and a fraction is injected into HPLC (Injection 3, HPLC conditions No. 4, FIG. 15). The particles are then washed twice with 200 μL of PBS. 20 μL of dithiothreitol (DTT) in 100 mM solution in PBS are then added to the medium, which is left under gentle stirring at 40° C. for one hour.

After magnetisation, the supernatant is injected into HPLC (Injection 4, HPLC conditions No. 4, FIG. 15).

Results and Conclusion:

On injection 1, the initial mixture with 44% ORN(RT=3 mins) and 56% ODN(RT=4 min) can be observed.

On injection 2, it is observed that only the initial ORN was functionalised (95%) by the BiotPEG₄(SS)IA Me since it has practically disappeared in favour of a cluster situated between 9 and 22 mins and corresponds to the acylated and biotinylated ORN. In contrast, it is noted that the ODN is still present and has not reacted.

Injection 3 demonstrates that the supernatant predominantly comprises ODN, with the functionalised RNA having been captured almost totally. The specific capture yield is estimated in this case at 37%, but it is evident that a further addition of streptavidin-coated magnetic particles would make it possible to capture the totality of this biotinylated RNA.

Finally, on injection 4, the total disappearance of the peaks corresponding to ORN and to ODN is observed. The cluster which corresponded to the acylated and biotinylated ORN(RT=9-32 mins) has also disappeared in favour of a cluster with a retention time principally between 4.5 and 18 mins coming from the cleaving of the disulfide bond (SS), and corresponding therefore to the loss of the biotin part. As for injection 2, the width of the cluster originates from the random functionalisation on the ORN. This induces the formation of a multitude of adducts with different retention times. In total, the ORN was freed from the support with a yield of 18% relative to the quantity involved at the start. It is possible to adjust the functionalisation rate in order to optimise this yield.

As an indication, in optimised conditions, the yield from the functionalisation/capture/elution process is around 50%.

A total hydrolysis of the biotinylated RNA by anthranilate SH groups, as indicated on the injection 4, was subjected to complete hydrolysis by Nuclease P1 and alkaline phosphatase. We have then shown that besides the four natural ribonucleosides, it was possible to observe nucleoside adducts and nucleotide anthranilate SH at the level of 5% of the total mixture. No trace of deoxyribonucleosides or nucleotides was observed.

This experiment confirms the specificity of the functionalisation/capture/cleaving/elution process, and therefore the possibility, starting from an ORN/ODN mixture, to selectively sift out the one or the other of these compounds according to the use which is to be made of them. The released acylated RNA is free from RNA, as is demonstrated by the total hydrolysis.

### EXAMPLE 12

### Demonstration of a Selective Method of Functionalising, Capturing, Cleaving and Eluting HIV WT (Wild Type) Transcripts with 1083 Nucleotides Reacted with Compound 5 Biot PEG₄ (SS) IA Me (24)

Objective:

In this example, we demonstrate that it is possible to:
selectively functionalise a population of amplifiable RNA strands (HIV WT transcript with 1083 nucleotides) by the compound 5 Biot PEG₄ (SS) IA Me (24),
selectively capture this population using streptavidin-coated magnetic particles,
chemically and specifically cleave the link connecting the immobilised RNA to the particle,
selectively elute the HIV transcript RNAs which have been subjected to the method as is described in FIG. 16.

Operating Mode:

We have an HIV transcript RNA with 1083 nucleotides, of which the partial sequence (insert pG3O) is in the bioMérieux HIV kit (Nuclisens EasyQ® HIV-1 v2.0, ref. 285033, bioMérieux, Marcy l'Etoile, France).

This transcript in solution in water, at 3.27*10¹² copies/μL (1.94 μg/μL), is produced internally.

Functionalisation:

The following reagents are introduced into five 250 μL polypropylene “PCR” tubes as indicated in Table 6 below:

TABLE 6
Summary of experimental functionalisation conditions (Ci: concentration
of the 5 Biot PEG₄ (SS) IA Me (24) stock solution, Cf: concentration of
BiotPEG₄(SS)IA Me (24) in the reaction mixture)

| | HIV transcript | | | 480 mM 5BiotPEG4(SS)IA | | |
|---|---|---|---|---|---|---|
| | RNA at | TEAAc buffer pH 7 | | Me (24) in DMF | | |
| | 1.94 μg/μL | conc. | vol. | Ci | vol. | Cf |
| Exp. | vol. (μL) | (M) | (μL) | (mM) | (μL) | (mM) |
| #1 | 2 | 1 | 1 | 0 | 1 | 0 |
| #2 | 2 | 1 | 1 | 60 | 1 | 15 |
| #3 | 2 | 1 | 1 | 120 | 1 | 30 |
| #4 | 2 | 1 | 1 | 480 | 1 | 120 |
| #5 | 2 | 2 | 0.5 | 480 | 1.5 | 180 |

For each of the experiments from Table 6, the total volume of the reaction mixture is 4 μL and the final TEAAc concentration is 250 mM. Experiment #1 acts as the control.

The filled tubes are incubated with the thermocycler (Thermoelectron Corporation, Milford, Mass., USA) for 1 h at 65° C.

Elimination of the Excess of 5 Biot PEG₄ (SS) IA Me (24) Biotinylating Reagent with the Nuclisens easyMAG Kit:

This step consists in eliminating the excess of 5 Biot PEG₄ (SS) IA Me (24) which has not reacted with the RNA. The Nuclisens easyMAG extraction kit from bioMérieux is used to do this.

After incubation, the functionalised samples are taken up in 100 μL of extraction buffer 1 (bioMérieux ref.: 280131, batch Z012EB1EB) and transferred into 1.5 mL polypropylene tubes which are autoclaved in advance. A quantity of 800 μL of this same buffer is then added before stirring and light centrifuging. A volume of 50 μL (i.e. 1 mg) of easyMAG magnetic silica particles (MagSil, bioMérieux ref.: 280133 batch ZO11EA1MS) is then added into each sample.

After incubation for 10 minutes at ambient temperature under gentle vortex stirring, the tubes are placed on a magnetic rack and the supernatant is removed. The 500 μL of extraction buffer are added before stirring, light centrifuging, magnetisation and elimination of the supernatant. Then 900 μL of extraction buffer 2 (ref.: 280131, bioMérieux, Lyon, France), 500 μL of extraction buffer 2 and 500 μL of extraction buffer 3 (ref.: 280132, bioMérieux, Lyon, France) are added successively, with stirring, light centrifuging, magnetisation and elimination of the supernatant between each addition systematically. Finally, 20 μL of extraction buffer 3 are added, and the tubes are placed in the thermomixer at 70° C. for 5 mins (1400 rpm stirring).

The eluate is measured with the Nanodrop ND-3300 (NanoDrop Technologies Inc, Wilmington, Del., USA) and injected with the BioAnalyzer onto Agilent RNA Nano 6000 chip (Agilent, Santa Clara, USA) to monitor its electrophoretic profile.

Capture:

100 μg (20 μL of solution) of MagPrep-25 streptavidin particles (Merck, Darmdstadt, Germany), washed beforehand twice with 200 μL of PBS 1x (0.01 M PO₄⁻, 0.0027 M KCl, 0.137 M NaCl, pH=7.4 at 25° C., SIGMA ref. 4417), is introduced into five new 250 μL (PCR-type) polypropylene tubes. To this is added 5 μL of a solution of PBS 4x+0.4% SDS (Sodium dodecyl sulphate), in order to limit the non-specific adsorption of the unfunctionalised RNAs, and then 15 μL of the eluate which emerges from the purification by easyMAG silica.

Each of the tubes is stirred at low speed (vortex) for 10 minutes at ambient temperature. The tubes are placed on a Dynal MPC 9600 magnetised rack (Dynal, Norway), and the supernatant is sampled carefully for Nanodrop assay (NanoDrop Technologies Inc., Wilmington, Del., USA), and injection into the BioAnalyzer Nano 6000 (Agilent, Santa Clara, USA) in order to measure a capture rate.

Elution:

The content of the tubes used in the preceding step is washed with 100 μL of PBS 4x+0.4% SDS solution, and then washed twice with 100 μL of a solution of PBS 1x (0.01 M PO₄, 0.0027 M KCl, 0.137 M NaCl, pH=7.4 at 25° C., SIGMA ref. 4417). The supernatants are removed carefully after magnetisation and 8 μL of 100 mM dithiothreitol (DTT) in PBS 1x pH 7.4 (SIGMA ref. 4417) are added to each of the tubes. The tubes are then separated and placed in the thermomixer (Eppendorf, Hamburg, Germany) for 1 h at 40° C. (300 rpm stirring). Finally, the tubes are placed onto the magnetised rack, and the supernatants are sampled for injection into the BioAnalyzer in order to measure an elution yield.

Results:

Functionalisation:

After functionalisation of the RNA with different concentrations of BiotPEG₄(SS)IA Me (24), there is observed on the electrophoretograms given by the BioAnalyzer a shift to the right of the functionalised RNA, as well as an enlargement of the peaks, as compared to the unfunctionalised control RNA. This is explained by the presence of the anthranilate residues, which lead to a growth of the mass of the RNA, and therefore to a modified migration profile, as shown in FIG. 17 or 18 (where line L corresponds to a scale of size of the nucleic acids, and lines 1, 2, 3, 4 and 5 correspond respectively to the transcript functionalised with molecule 24 at 15, 30, 60, 120 or 180 mM). The more the RNA is functionalised, the more it is shifted to the right or upward, which expresses an increase of the functionalisation rate.

Capture:

After capture of the functionalised RNA transcripts, with different concentrations of BiotPEG₄(SS)IA Me (24), RNA is detected solely in the supernatant of the control and in very low quantity in the supernatant of experiment #2 (functionalisation with 15 mM BiotPEG₄(SS)IA Me (24)), as shown in the electrophoretograms of FIG. 19. This indicates that the RNA is captured fully when it has been sufficiently biotinylated. Since the control in experiment 1 is not biotinylated, it is therefore not captured.

Elution:

After elution of the transcripts immobilised in the preceding step, RNA is detected significantly in all of the samples, except in the control as expected. Since the latter has been neither functionalised nor captured, it is therefore not eluted. At the same time it is demonstrated that there is no non-specific elution due to unbiotinylated RNA which would be adsorbed on the particles. The quantity of RNA eluted is dependent upon the functionalisation rate, as shown in FIG. 20. It is noted that the RNA thus eluted has a shorter retention time than that of the functionalised RNA, this is due to the cutting of the link SS, which makes it possible to reduce the molecular weight of the molecule.

The respective yields of the process and of each of its different steps are summarised in Table 7 below:

TABLE 7
Summary of the yields obtained during different steps of
functionalisation, capture and elution of an HIV transcript reacted
with the compound 5BiotPEG₄(SS)IA Me (24)

| | % RNA recovered after | | | |
|---|---|---|---|---|
| | elimination of the | Capture | elution | overall |
| Experiments | excess of reagent 24. | rate | rate | yield |
| #1 | 35% | 6% | nd | 0% |
| #2 | 32% | 77% | 40% | 10% |
| #3 | 34% | 100% | 53% | 18% |
| #4 | 31% | 100% | 45% | 14% |
| #5 | 30% | 100% | 43% | 13% |

All of the concentrations were measured by integrating the fluorescence signals obtained with the BioAnalyzer. The capture rate is defined as the ratio between the quantity of RNA which has been immobilised on the MagPrep-25 Merck Streptavidin particles (evaluated by assaying the RNA in the supernatant) and the quantity of RNA recovered after functionalisation and precipitation.

The elution rate is deducted from the yields of each of the steps, and from the overall yield.

Overall yields of between 10% and 18% are obtained for the different concentrations of BiotPEG₄(SS)IA Me, with the understanding that between 30% and 35% of the material is recovered after purification with the Nuclisens easyMAG kit. Starting from a BiotPEG₄(SS)IA Me (24) concentration of 30 mM and beyond, the capture is total with 100 μg of MagPrep-25 Streptavidin particles (Merck).

The non-specific adsorption of unfunctionalised RNA (control) on the MagPrep-25 Streptavidine particles is 6%, but this RNA is eliminated during washings and is not eluted during treatment with DTT 100 mM.

The handling was duplicated for each of the concentrations with similar results.

Conclusion:

The HIV transcript RNAs were functionalised with the compound BiotPEG₄(SS)IA Me (24) at different concentrations, purified with the Nuclisens easyMAG® kit, captured selectively on MagPrep-25 (Merck) Streptavidin magnetic particles and eluted by cleaving the disulfide bond.

Only the RNA which has been functionalised is detected with the BioAnalyzer (Agilent RNA 6000 Nano chip).

Up to 18% of the starting RNA was successfully extracted via this method.

This unambiguously demonstrates that it is possible to perform specific functionalisation of the RNA and its elution after capture on magnetic particles.

### EXAMPLE 13

### NASBA Amplification of HIV Transcripts Functionalised Selectively with the Compound BiotPEG₄(SS)IAMe (24), Captured with Streptavidin-Coated Magnetic Particles and Eluted with a 100 mM DTT Solution.

Objective:

We demonstrate the ability of the transcripts prepared in example 12 to be amplified subsequently during an amplification reaction; in the present case it is the NASBA amplification technique, but this can be carried out with other techniques such as PCR, TMA, 3SR, etc. We also show that the presence of an anthranilate residue on the RNA does not hamper the amplification.

Operating Mode:

In the preceding example 12, 4 μg of a WT HIV transcript (1083 nucleotides, internal production, bioMérieux batch 841470301, C=3.27E12 copies μl) was biotinylated with different concentrations of the compound (24) (0 mM, 15 mM, 30 mM, 120 mM, 180 mM, i.e. the experiments #1-7 of Table 6). This transcript was then captured on Merck MagPrep 25 Streptavidin magnetic particles, then, after rinsing, the particles were subjected to a 100 mM DTT treatment in PBS 1x pH 7 at 40° C. for one hour, in order to cleave the disulfide bond (SS) and elute the transcripts with —SH anthranilate groups, as shown in FIG. 16. We evaluated the ability of these transcripts to be amplified by the NASBA technology. This evaluation was performed as a comparison with an unfunctionalised “bare” HIV_WT transcript range.

The amplification is performed on a NucliSENS EasyQ instrument (bioMérieux, Lyon, France) and with a NucliSENS EasyQ HIV-1 V2.0 kit (bioMérieux, Lyon, France, ref. 285033, batch 09122106). The software used is NucliSENS EasyQ Director, and the protocol is QL1-60 1.0 (bioMérieux, Lyon, France).

After functionalisation with compound 24, capture and elution in 8 μL of 100 mM DTT, as described in example 12, the transcripts of experiments #2-5 from Table 7 were assayed on RNA 6000 NANO chip with the AGILENT bioanalyzer (Santa Clara, USA), and were diluted in the following concentrations: 1000 copies, 100 copies, 50 copies, 10 copies, 1 copy, 0 copies in 15 μl. An HIV IC transcript (internal control of the same amplified sequence as the WT (wild type) transcript, but detected by a molecular beacon carrying another fluorophore) diluted to 360 copies is added to the 15 μl volume, and will be present in all of the wells in which the amplifications occur. This transcript serves to monitor the correct operation of the amplification. The tests are carried out in triplicate.

Experiment #1 corresponds to a transcript which was not functionalised (0 mM of compound (24) but which has nevertheless undergone all the steps of the functionalisation, capture and elution protocol. This therefore is to control the non-specific adsorption and elution of an unfunctionalised transcript. Since the quantity of RNA contained in this sample is lower than the bioAnalyzer's detection threshold, it is assayed by NASBA amplification. Table 8 below indicates the assay values of the eluates of each transcript of experiments #1-5 (total volume=8 μl).

TABLE 8
Assay values of the eluted transcripts produced in example 12

| | | Concentration of | | |
|---|---|---|---|---|
| | Concen- | functionalised/ | Total eluted | Total yield |
| | tration of | captured and | quantity | functionalisation/ |
| | compound | eluted transcript | (in the | capture/elution of |
| Exp. | 24 (mM) | (in copies/μl) | 8 μl) | the HIV transcripts |
| #1 | 0 | 6.7E+04 | 0.0000003 | 0.00001% |
| #2 | 15 | 7.6E+10 | 0.3609520 | 9.30289% |
| #3 | 30 | 1.4E+11 | 0.6649116 | 17.13690% |
| #4 | 120 | 1.0E+11 | 0.4844356 | 12.48545% |
| #5 | 180 | 9.8E+10 | 0.4654381 | 11.99583% |

Six Accuspheres ENZ II (NucliSENS EasyQ HIV-1 V2.0 kit, ref. 285033, bioMérieux, Lyon, France) are deposited into a 1.5 ml polypropylene tube. A volume of 270 μL of ENZdil H (NucliSENS EasyQ HIV-1 V2.0 kit, ref. 285033, bioMérieux, Lyon, France) is added onto the Accuspheres, and 15 minutes are allowed to pass, for them to dissolve completely. The twelve Accuspheres PRM H (NucliSENS EasyQ HIV-1 V2.0 kit, ref. 285033, bioMérieux, Lyon, France) are deposited into a 1.5 ml polypropylene tube 1080 μL of PRMdi l H (NucliSENS EasyQ HIV-1 V2.0 kit, ref. 285033, bioMérieux, Lyon, France) is added onto these Accuspheres and the mixture is immediately vortex stirred until the Accuspheres dissolve completely. After complete dissolution in the two tubes (mix ENZ II and mix PRM H), these mixtures are centrifuged with a benchtop centrifuge.

The NASBA amplification is performed in well strips of 8 wells of 0.2 ml sealed by their stopper. The 15 μL volume of mixture of transcripts at each concentration (between 1000 and 1 copies) is deposited at the bottom of each well. 20 μL of PRM H mix is added. The well strips without stopper are deposited onto a NucliSENS EasyQ Incubator (bioMérieux, Lyon, France) following the NASBA RNA protocol for 2 minutes at 65° C. (denaturing of the RNAs), then for 2 minutes at 41° C. (amplification temperature). During this time, 5 μL of ENZ II mix are deposited into the stoppers of the well strips. The well strips are then stoppered, centrifuged with the benchtop centrifuge, and then stirred for 3 seconds, and once again centrifuged with the benchtop centrifuge, before being deposited onto the rack of the NucliSENS EasyQ instrument (bioMérieux, Lyon, France). In parallel, the same operation is performed with a range of non-functionalised HIV_WT transcripts from 1000 to 1 copy (copies). The amplification then starts. After 60 minutes, the results are exported in .xml format and analysed using the SpotFire software (Tibco Softaware Inc., Palo Alto, USA) with the T2 algorithm, such as can be seen in FIG. 21.

Results and Conclusion:

As shown in Table 8, it is noted that the control sample representing the adsorption and non-specific elution (exp #1) contains practically no transcripts and, on average, it contains 1 million times fewer than the samples corresponding to experiments #2-5. It may therefore be said that the method of functionalisation, capture, cleaving and elution of an HIV transcript reacted with compound (24) is an RNA-specific method, since an RNA transcript which has not reacted with compound (24), but which has followed the same steps, is only eluted with a concentration 1 million times lower. It is therefore evident that the NASBA amplification reactions observed in FIG. 21 may only come from the HIV_WT transcript functionalised, captured and eluted according to the process described in example 12.

We observe (not shown) an amplification of the internal control with 360 copies at all points in accordance with expectations, which signifies that the elution buffer (PBS 1x pH 7 and DTT 100 mM), diluted to the concentration necessary to perform the range of transcripts from 1000 to 1 copy(copies), does not inhibit the NASBA amplification.

FIG. 21 shows a superposition of ranges of HIV transcripts (1000, 100, 50, 10 and 1 copy (copies)) functionalised with molecule (24) at different concentrations (15, 30, 120 and 180 mM), captured on streptavidin-coated magnetic particles, then eluted by chemical cleaving with DTT and amplified by NASBA. A comparison is carried out with a range of unbiotinylated transcripts.

For concentrations of 15 and 30 mM, the NASBA amplification is not impacted. In contrast, from 120 mM, we observe incipient inhibition of the amplification, which is confirmed with a label concentration of 180 mM, with a more and more substantial time offset in the start of the amplification. This is explained by the fact that the more the RNA is functionalised, the more the anthranilate units may hamper hybridisation and/or the polymerase elongation process.

The NASBA amplification process generally holds up, but an increase is observed in the minimal quantity of transcript detected when the concentration of functionalisation reagents (24) increases, as is indicated in Table 9 below.

TABLE 9
Minimum number of copies of HIV transcripts functionalised,
captured, eluted and detected as a function of the concentration
of functionalisation reagent (24)

| Concentration of | Minimum number of |
|---|---|
| compound 24 (mM) | copies detected |
| 0 | 10 |
| 15 | 10 |
| 30 | 10 |
| 120 | 100 |
| 180 | 1000 |

It is therefore good to moderate the functionalisation of the RNA so as to avoid inhibition of the amplification, even if functionalisation is possible even at high concentration. In an embodiment of the invention, 50 mM of compound (24) remains a preferred upper limit to be used in the conditions which we have in this example.

This demonstrates that RNAs may be subjected to a process of functionalisation, capture and elution, whilst keeping their ability to be amplified by an enzymatic polymerisation reaction.

As a further remark, it should be noted that a strong functionalisation rate is also advantageous. Thus, this situation makes it possible to inhibit the amplification of RNA without affecting that of DNA. This may find application in approaches for decontaminating solutions polluted by RNA.

### EXAMPLE 14

### Selective Extraction of HIV RNA Transcripts from a Solution Containing a Mixture of HIV RNA Transcripts and Genomic DNA.

Objective:

We are demonstrating the concept of DNA enrichment, of a biological solution containing a mixture of RNA and DNA. We are testing the concept on three different biological RNA models, and we are demonstrating that the selective process:
of functionalising the RNA with compound 24,
of selectively capturing using streptavidin-coated magnetic particles, and
of cleaving/eluting (such as described in example 12)
   makes it possible to go from an initial RNA/DNA ratio of 20/80 to a final RNA/DNA ratio of 90/10 on average.

Operating Mode:

The nucleic acids used in this example are as follows:
Reference total RNAs (Human Universal Reference RNA, MAQC-A, STRATAGENE ref. 740000 (Santa Clara, USA)) at 1150 ng/μl.
HIV transcript (so-called Wild Type or WT) at 1.94 μg/μl.
calf thymus gDNA, SIGMA (Saint Louis, USA), ref. D4764-5UN, at 1.76 μg/μl.
EasyMag® eluate (437 ng/μl with 12% RNA/88% DNA) obtained after extraction of 200 μL of blood using the Nuclisens EasyMAG® extraction kit from bioMérieux (Marcy l'Etoile, France), as described in example 12.

1—Functionalisation:

The following reagents are respectively introduced (Table 10 below) into three 0.2 ml plastic tubes:

TABLE 10
Summary of experimental conditions described in example 14

| | | | calf | | | 6 mM | |
|---|---|---|---|---|---|---|---|
| | MAQC-A | HIV WT | thymus | EasyMag | 1M TEAAc | BiotPEG4(SS)IAMe | |
| | RNA | transcript | gDNA | eluate | pH 7 | (24)/DMF | |
| | Vol. | Vol. | Vol. | Vol. | Vol. | Vol. | DNA/RNA |
| Tests | (μL) | (μL) | (μL) | (μL) | (μL) | (μL) | ratio |
| 1 | | 1 | 4.5 | | 2.8 | 2.8 | 80/20 |
| 2 | 1.7 | | 9.1 | | 5.4 | 5.4 | 89/11 |
| 3 | | | | 22.9 | 11.5 | 11.5 | 88/12 |

In each case, the final TEAAc concentration is 250 mM and the final concentration of compound 24 is 1.5 mM.

The three tubes are incubated for 1 hour at 65° C. on a heating rack.

2—Elimination of the Excess of Compound 24 by Isopropanol/Acetate Precipitation

The totality of the volume of each test is transferred into autoclaved 1.5 ml plastic tubes. The volume in each tube is made up to 100 μl with ultrapure water. 30 μL of 3M sodium acetate (pH=5.6), and then 70 μL of isopropanol (Sigma ref. 34959 (St Louis, USA)), are added. All of the tubes are stirred by vortex effect, and then centrifuging is carried out for 30 minutes at 14,000 RPM at +4° C. At the end of centrifuging, the supernatant is removed using tapered tips. The pellet is rinsed with 50 μL of 70% ethanol, and then centrifuging is carried out for 15 mins at 14,000 RPM at +4° C. After the centrifuging, the supernatant is removed by turning over the tubes. The pellets contained in the tubes are then dried for 10 minutes with the rotary evaporator. The pellets are taken up with 20 μL of ultrapure water. The nucleic acids recovered in the supernatants of each tube are assayed with the Qubit® Fluorometer instrument, ref. Q32857, Invitrogen (Carlsbad, Calif.), and using the Quant-iT RNA Assay Kit 5-100 ng, ref. Q32855, Invitrogen (Carlsbad, Calif.), and Quant-iT dsDNA HS Assay Kit 0.2-100 ng, ref. Q32854, Invitrogen (Carlsbad, Calif.). These assay kits make it possible to determine an RNA/DNA ratio in a mixture.

3—Capture of the Nucleic Acids on the Streptavidin-Coated Magnetic Particles:

Taking into account the substantial quantity of nucleic acids in each tube, 60 μL of ultrapure water are added. The capture of the nucleic acids for each test is performed in five different tubes (division of the volume into five tubes), so as to achieve the optimal conditions for this step (i.e. that 40 μg of magnetic particles fully capture 2 μg of nucleic acids). For each test, five 0.2 ml plastic tubes are used, into which 8 μl (equivalent to 40 μg) of MagPrep P-25 Streptavidin, MERCK (Darmstadt, Germany) is added. The particles are washed twice with 80 μL of PBS 1X+SDS 0.1%, using tapered tips and the Invitrogen MPC 9600 Dynal magnet (Carlsbad, Calif.) for the magnetic separations. Once the particles are washed, 15 μL of purified nucleic acids prepared in step 2 and 5 μL of PBS 4X+SDS 0.4% are added onto each pellet. The tubes are incubated for 10 minutes with gentle stirring (vortex stirrer at minimum speed) at ambient temperature. After 10 minutes, the supernatant is sucked up after magnetic separation using the magnet and tapered tips. This supernatant is assayed with the Qubit® Fluorometer as previously.

4—Elution of the Functionalised RNAs:

The pellets of streptavidin magnetic particles, on which the functionalised nucleic acids are immobilised, are washed with 80 μL of PBS 1X/SDS 0.1% for 5 minutes at 65° C. (heating rack). Vortex stirring is performed and after 5 minutes, the wash buffer is sucked up after magnetic separation using the magnet and tapered tips. This operation is repeated twice. A last washing of the pellets is performed with 20 μl of PBS 1X. The five pellets corresponding to each test are then mixed in a single tube. The volume is then 100 μl of PBS 1X for each test. Vortex stirring is performed, then the supernatant is sucked up after magnetic separation using the magnet and tapered tips. 8 μl of DTT 100 mM/PBS 1X is added onto each pellet. Vortex stirring is performed, and incubation is performed for 1 hour at 40° C., 300 RPM. After 1 hour, the supernatant is sucked up, after magnetic separation using the magnet and tapered tips. This supernatant will be assayed with the Qubit® Fluorometer (Invitrogen (Carlsbad, Calif.) instrument as previously.

Results and Conclusions:

1—Results:

The assays with the Qubit® Fluorometer of the eluates make it possible to quantify the nucleic acids present at each step of the process as described previously.

From the measurements obtained, it is possible to calculate the RNA and DNA extraction yields, then a selectivity index known as S, which makes it possible to conduct a comparison of several tests performed under different conditions:

$$S = \frac{RNA extraction Yld}{DNA extraction Yld} = \frac{{[ RNA ]}_{f} / {[ RNA ]}_{i}}{{[ DNA ]}_{f} / {[ DNA ]}_{i}}$$

The following table 11 is drawn up from the measurements obtained and calculations performed:

TABLE 11
Summary of the DNA/RNA ratios measured at step 1
(initial) and at step 4 (final) of the RNA functionalisation/
capture/cleaving/elution process

| | DNA/RNA | DNA/RNA | |
|---|---|---|---|
| | ratio | ratio | Selectivity |
| Models | (initial) | (final) | S |
| HIV RNA Transcript/calf | 80/20 | 9/91 | 26 |
| thymus gDNA | | | |
| MAQC RNA/calf thymus gDNA | 89/11 | 14/86 | 50 |
| Easy MAG eluate from total | 88/12 | 12/88 | 54 |
| blood | | | |

The results obtained are similar for the three biological models. From a mixture of DNA and RNA on average comprising 11-20% of RNA, and under the experiment conditions described in the operating mode, it is possible to enrich the mixture up to 86-91% RNA. The selectivity is in every case far greater than 1.

2—Conclusion:

The concept of RNA enrichment after the process of selective functionalisation of RNA with compound 24, selective capturing using streptavidin-coated magnetic particles, and of cleaving/elution (as described in example 12) is demonstrated on three different biological models. In every case, a very significant enrichment of the RNA solution is clearly demonstrated. The technique described in this invention application is to our knowledge the only one which makes it possible to perform this operation of selectively sorting RNA in a DNA/RNA mixture.

### EXAMPLE 15

### Selective Extraction of Genomic DNA from a Solution Containing a Mixture of HIV RNA Transcripts and Genomic DNA

Objective:

We demonstrate the concept of DNA enrichment of a biological solution containing a mixture of RNA and DNA. We demonstrate that the selective process:
of functionalising RNA with compound 24,
then selectively capturing RNA functionalised using streptavidin-coated magnetic particles,
   makes it possible to obtain a supernatant free from RNA, since the initial RNA/DNA ration of 14/86 becomes equal to 0/100 after this process.

Operating Mode:

The nucleic acids used in this example are as follows:
HIV_WT transcript, at 1.94 μg/μl.calf thymus gDNA, SIGMA (Saint Louis, USA), ref. D4764-5UN, at 1.76 μg/μl.

1—Functionalisation:

With these two nucleic acid solutions, a 14%/86% RNA/DNA mixture is prepared with the following composition: 6.2 μL of HIV RNA transcript (i.e. 12 μg) and 27.3 μL of genomic DNA (i.e. 48 μg). The following reagents according to table 12 below are introduced into 0.2 ml plastic tubes:

TABLE 12
Summary of experimental conditions described in example 15

| | RNA/DNA | | | |
|---|---|---|---|---|
| | mixture | 1M TEAAc | BiotPEG4(SS)IA | |
| | (14/86) | pH 7 | Me (24)/DMF | DMF |
| Tests | Vol. (μL) | Vol. (μL) | Vol. (μL) | Vol. (μL) |
| 1 | 6 | 3 | | 3 |
| 2 | 6 | 3 | 3 μl at 6 mM | |
| 3 | 6 | 3 | 3 μl at 60 mM | |
| 4 | 6 | 3 | 3 μl at 120 mM | |
| 5 | 6 | 3 | 3 μl at 240 mM | |

In every case, the final concentration of TEAAc is 250 mM and the final concentration of compound 24 tested is 0, 1.5, 15, 30 and 60 mM.

The five tests are incubated for 1 hour at 65° C. on a heating rack.

2—Elimination of the Excess of Compound 24 by Purification on EasyMAG® Magnetic Silica:

Taking account of the significant quantity of nucleic acids in each tube, the purification for each test is performed in five 1.5 ml plastic tubes. Therefore 2.2 μl (i.e. 2 μg of nucleic acids) is sampled five times for each test following the EasyMag® purification protocol (bioMérieux, Marcy l'Etoile, France) as described in example 12. For this, 900 μL of “lysis buffer” (extraction buffer 1, ref. 280130, batch Z012EB1EB, bioMérieux) is added into each tube. Stirring by vortex effect, and brief centrifuging are performed. 50 μL of magnetic silica particles, i.e. 1 mg (EasyMAG silica, ref. PR333077, batch Z011MA1MS, bioMérieux) is added. Stirring by vortex effect is performed immediately. The tubes are incubated for 10 minutes at ambient temperature and without stirring. After 10 minutes, the supernatant is sucked up after magnetic separation using the DYNAL Invitrogene magnet (Carlsbad, Calif.). 500 μL of “lysis buffer” is added, stirring by vortex effect is performed, followed by magnetisation and elimination of the supernatant. 900 μl of wash buffer 2 (extraction buffer 2, ref. 280131, batch Z011LD2EB, bioMérieux) is added, stirring by vortex effect is performed, followed by magnetisation and elimination of the supernatant. 500 μL of wash buffer 2 is added, vortex stirring is performed, followed by magnetisation and elimination of the supernatant. 500 μl of wash buffer 3 (extraction buffer 3, ref. 280132, batch Z011HD3EB, bioMérieux) are added, vortex stirring is performed, followed by centrifuging, magnetisation and elimination of the supernatant. 20 μL of wash buffer 3 is added, mixing is performed by tapping the tube and elution is performed in a heating rack for 5 minutes at 70° C., with stirring at 1400 rpm. Centrifuging, magnetisation and elimination of the supernatant are performed. The nucleic acids recovered in the supernatants are assayed with the Qubit® Fluorometer and the corresponding kits as described in example 14.

3—Capture of the Nucleic Acids on the Streptavidin-Coated Magnetic Particles:

For each test, five 0.2 ml plastic tubes are used, into which 8 μl (equivalent to 40 μg) of MagPrep P-25 Streptavidin, batch FA007950 841, MERCK (Darmstadt, Germany) is added. The particles are washed two times with 80 μL of PBS 1X+SDS 0.1%, using tapered tips and the Dynal MPC 9600 magnet for the magnetic separations. Once the particles have been washed, 15 μL of the supernatant recovered after purification (step 2) and 5 μL of PBS 4X+SDS 0.4% are added onto each pellet. The tubes are incubated for 10 minutes with gentle stirring (vortex at minimum speed) at ambient temperature. After 10 minutes, the supernatant is sucked up after magnetic separation using the magnet and tapered tips. This supernatant is assayed with the Qubit® Fluorometer in order to determine the RNA/DNA ratios.

Results and Conclusions:

1—Results:

The assays with the Qubit® Fluorometer make it possible to quantify the nucleic acids present at each step of the process. From these measurements obtained, it is possible to calculate the DNA/RNA ratios at each step. The data are recorded in Table 13 below:

TABLE 13
Summary of the values of the DNA/RNA ratios measured
during steps 1 (column 2) and 3 (column 3) of the
RNA functionalisation/capture process

| | | 3 |
|---|---|---|
| | 2 | DNA/RNA (final in |
| 1 | RNA/DNA | the supernatant after |
| [24] | (initial) | capture of the RNA) |
| 0 mM | 86/14 | 34/66 |
| 1.5 mM | 86/14 | 38/62 |
| 15 mM | 86/14 | 100/0 |
| 30 mM | 86/14 | 100/0 |
| 60 mM | 86/14 | 100/0 |

From a mixture comprising approximately 85% DNA, and starting from the use of compound 24 at 15 mM, it is possible to assay 100% of DNA in the capture supernatant (biotinylated RNA having been selectively captured on the streptavidin magnetic particles).

2—Conclusion:

We demonstrate by this example that it is possible to selectively biotinylate RNA in the presence of 80% DNA with the isatoic compound 24, and then to immobilise the biotinylated RNA with streptavidin-coated magnetic particles in order to have a supernatant comprising to 100% DNA This technique advantageously replaces an RNAse.

1. A process of functionalising at least one ribonucleic acid (RNA) molecule, which comprises the following steps:
a) having at least:
a binding molecule constituted by an isatoic anhydride or a derivative thereof,
a group of interest, and
a binding arm linking the binding molecule with the group of interest,
b) reacting the anhydride function of the binding molecule with at least one hydroxyl group in:
position 2′ of the ribose of one of the RNA nucleotides, and/or
position(s) 2′ and/or 3′ of the ribose of the nucleotide at the 3′ terminal end of the RNA, thereby
c) obtaining an anthranilate linking, via the binding arm, the RNA to the group of interest.

2. The process of claim 1, wherein the functionalising comprises labelling at least one ribonucleic acid (RNA) molecule, and comprises the following steps:
a) having at least:
a binding molecule constituted by an isatoic anhydride or a derivative thereof, which has an intrinsic fluorescence,
a group of interest, which has an intrinsic fluorescence signal, but which is different from the signal emitted by the binding molecule, or which does not have an intrinsic fluorescence signal, and
a binding arm linking the binding molecule with the group of interest,
b) reacting the anhydride function of the binding molecule with at least one hydroxyl group in:
position 2′ of the ribose of one of the RNA nucleotides, and/or
positions 2′ and/or 3′ of the ribose of the terminal nucleotide in position 3′ of the RNA, and
obtaining an anthranilate linking, via the binding arm, the RNA to the group of interest.

3. The process of claim 1, wherein the functionalising comprises capturing or separating at least one ribonucleic acid (RNA) molecule, and comprises the following steps:
a) having at least:
a binding molecule constituted by an isatoic anhydride or a derivative thereof,
a group of interest constituted by a ligand which is complementary to an anti-ligand, and
a binding arm linking the binding molecule with the group of interest,
b) reacting the anhydride function of the binding molecule with at least one hydroxyl group in:
position 2′ of the ribose of one of the RNA nucleotides, and/or
positions 2′ and/or 3′ of the ribose of the terminal nucleotide in position 3′ of the RNA,
c) obtaining an anthranilate linking, by means of the binding arm, the RNA to the group of interest, and
d) capturing or separating RNA through a ligand—anti-ligand reaction.

4. The process, according to claim 1, wherein the binding arm is associated with the binding molecule before said binding arm is associated with the group of interest.

5. The process, according to claim 1, wherein the binding arm is associated with the binding molecule after said binding arm is associated with the group of interest.

6. The process, according to claim 1, wherein the binding molecule is associated with the RNA beforehand.

7. The process of claim 1, wherein the functionalising comprises selectively capturing at least one RNA molecule using at least one binding molecule, wherein the group of interest is constituted by a ligand which is complementary to an anti-ligand, and wherein the binding molecule is constituted by an isatoic anhydride or a derivative thereof, which attaches via a covalent bond to a hydroxyl group in:
position 2′ of the ribose of one of the RNA nucleotides, and/or
position 2′ and 3′ of the ribose of the terminal nucleotide in position 3′ of the RNA, and/or
position 3′ of the ribose of said terminal nucleotide in position 3′ of the RNA.

8. The process of claim 7, further compromising separating RNA molecules from other biological constituents, in particular DNA molecules, in a biological sample containing undifferentiated nucleic acids (RNA and DNA) comprising:
having the group of interest associated with at least one solid support which is easily separable from the rest of the biological sample, and
separating the binding molecules bearing the RNA molecules from the rest of the biological sample.

9. A functionalising reagent of the formula (1): 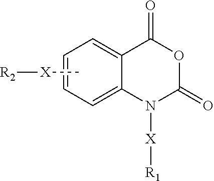
wherein:
R₁ represents H or a group of interest,
R₂ represents H or a group of interest which can be:
a. a label or a labelling precursor, or
b. a ligand able to be recognised by a recognition molecule or a surface, or a particle, etc., in order to form a stable complex,
if R₁ is represented by H, R₂ is represented by a group of interest, and vice-versa, and
X is a binding arm.

10. The reagent according to claim 9, wherein the functionalising comprises capturing or separating, and the reagent further comprises a capture or separation means comprising a solid support, a filter, or an inner wall of a receptacle.

11. A functionalising reagent according to claim 9, wherein the binding arm X comprises:
a single covalent bond linking an atom of the binding molecule and an atom of the group of interest, or
an organic binding arm, selected from
a single covalent bond between the binding molecule and the group of interest,
a single carbon atom, substituted or unsubstituted, and
a chain formation of at least two carbon atoms, optionally further comprising aromatic structures and/or heteroatoms (oxygen, sulfur, nitrogen, etc.).

12. A functionalising reagent according to claim 11, wherein the binding arm X further comprises a function or a bond capable of being cleaved by a physicochemical, photochemical, thermal, enzymatic and/or chemical means which separates the binding molecule from the RNA under particular light, temperature, enzymatic or chemical conditions.

13. A functionalised biological RNA molecule obtained by the process according to claim 1.

14. A kit for detecting a target RNA molecule, comprising a reagent according to claim 9.

15. The functionalising process according to claim 1, which comprises a supplementary step between steps a) and b), comprising hydrolysing the terminal monophosphate group in position 3′ of each RNA strand to be functionalised.

16. The reagent of claim 10 wherein the solid support comprises magnetic or non-magnetic polymer or silica particles.
